(19) United States
(12) Patent Application Publication
(10) Pub. No.: US 2025/0384958 A1
(43) Pub. Date: Dec. 18, 2025

(54) DOSE-RESPONSE MODEL FOR ACCURATE DETECTION AND QUANTIFICATION OF TRANSCRIPTOME-WIDE GENE KNOCKDOWN FOR OLIGONUCLEOTIDE-BASED MEDICINES

(71) Applicant: Creyon Bio, Inc., Carlsbad, CA (US)

(72) Inventors: Swagatam Mukhopadhyay, Carlsbad, CA (US); David Pekker, Carlsbad, CA (US); Steven G. Kuntz, Carlsbad, CA (US)

(21) Appl. No.: 19/217,994

(22) Filed: May 23, 2025

Related U.S. Application Data
(60) Provisional application No. 63/651,906, filed on May 24, 2024.

(51) Int. Cl. G16B 25/10; G16B 5/20; G16B 40/20; G16H 20/10

(52) U.S. Cl. CPC G16B 25/10; G16B 5/20; G16B 40/20; G16H 20/10

(57) ABSTRACT

The methods of the present disclosure include a dose-response model (DoReSeq) and machine learned models for quantifying oligonucleotide mediated off-target gene or on-target gene knockdown, and/or characterizing the level of gene expression dependent upon concentration of an oligonucleotide.

### 1. BACKGROUND

Synthetic antisense oligonucleotides and siRNAs are a class of Oligonucleotide-Based Medicines (OBMs) that can hybridize with pre-mRNA and mRNA, recruit a mechanism-of-action specific enzymatic complex, and knockdown target gene expression. This class of molecules provides an excellent substrate for designing precision gene-modulatory therapeutics; however, quantifying on- and off-target dose response as measured by next-generation sequencing for this class of therapeutics has remained under-powered and ambiguous. Often in silico predictions of off-targets (ranked by edit tolerance) are used as putative off-target analysis in ASO and siRNA drug design.

There is a need for methods of designing and optimizing oligonucleotide-based medicines that have strong affinity to on-target genes with minimal or no interaction with off-target genes. There is a need to utilize in silico and machine learning to understand and characterize gene expression dependent upon the concentration of an oligonucleotide and as detected by functional genomics analysis in order to: identify which genes are knocked down, and quantify and characterize on- and off-target genes in order to develop safe and effective oligonucleotide-based medicines.

### 2. SUMMARY

Synthetic antisense oligonucleotides and siRNAs are a class of Oligonucleotide-Based Medicines (OBMs) that can hybridize with pre-mRNA and mRNA, recruit a mechanism-of-action specific enzymatic complex, and knockdown target gene expression. This class of molecules provides an excellent substrate for designing precision gene-modulatory therapeutics; however, quantifying on and off-target dose response as measured by next-generation sequencing for this class of therapeutics as remained under-powered and ambiguous. Often in silico predictions of off-targets (ranked by edit tolerance) are used as putative off-target analysis in ASO and siRNA drug design. The present inventors constructed a simple, effective theory of transcriptional dynamics and enzymatic activity in order to describe the transcriptome-wide response to these oligonucleotides. The present inventors established rigorous quantification methods of off-target analysis in oligonucleotide drug design. The present inventors also extended the DESeq work of Negative Binomial noise in gene expression measurements to describe noise, including outliers, in OBM-dose response experiments. The present inventors demonstrated the performance of the model on both synthetic and experimental Digital Gene Expression (DGE) data of dose response in ASO-treated cells, to elevate the standards of off-target analysis for such an important class of precision therapeutics.

An aspect of the present disclosure includes method for manufacturing oligonucleotide-based medicines.

In some embodiments, the method includes administering a set of oligonucleotides to one or more cells, wherein each oligonucleotide within the set of oligonucleotides is administered at plurality of different dosages. In some embodiments, the method includes performing a functional genomic analysis operation on the one or more cells to quantify gene expression for each oligonucleotide dosage. In some embodiments, the method includes creating a training set by fitting a dose-responsive model to a dose-dependent response of each oligonucleotide on gene expression, wherein the dose-responsive model comprises a kinetic model of dose-response. In some embodiments, the dose-responsive model further comprises a noise model of gene expression. In some embodiments, the dose-responsive model further comprises a Bayesian inference model to detect and quantify dose-responsive gene features.

In some embodiments, the dose-responsive model is configured to identify and characterize dose-responsive gene features dependent upon the dosage of each oligonucleotide of the set of oligonucleotides.

In some embodiments, the method includes training a machine-learned model using the training set, the machine learned model configured to determine whether the dose-responsive gene feature is or is not associated with the oligonucleotide sequence.

In some embodiments, if the dose-responsive gene feature is determined to be associated with the oligonucleotide sequence, the machine-learned model is configured to further: identify properties of the oligonucleotide sequence or target genes that result in on-target gene knockdown above a threshold; ii. identify properties of the oligonucleotide sequence or target genes that are susceptible to off-target gene knockdown above a threshold; and/or ii. identify one or more target loci of target genes susceptible to on-target gene knockdown above the threshold.

In some embodiments, if the dose-responsive gene feature is determined to not be associated with the oligonucleotide sequence, the machine-learned model is configured to further: identify one or more target loci of target genes susceptible to on-target gene knockdown and/or off-target gene knockdown above the threshold.

In some embodiments, the method includes validating a second set of oligonucleotides by synthesizing the second set of oligonucleotides based on the machine-learned model, administering the second set of oligonucleotides to a second set of cells at different dosages, performing the functional genomic analysis operation on the second set of cells to quantify gene expression, and measuring a difference between the quantified gene expression and a predicted gene expression produced by the machine-learned model. In some embodiments, the method includes modifying the machine-learned model based on the measured difference between the quantified gene expression and a predicted gene expression produced by the machine-learned model.

In some embodiments, the functional genomic analysis operation is a transcriptome analysis. In some embodiments, the functional genomic analysis operation is a sequencing based analysis. In some embodiments, the transcriptome analysis is selected from: digital gene expression (DGE), RNA sequencing (RNA-seq), tag-based RNA-seq (TAQ-seq), or a combination thereof. In some embodiments, the functional genomic analysis is based off of NGS assays, DNA/RNA sequencing assays-based readouts, and/or protein-level readouts like proteomics and ELISA assays for corresponding RNAs.

In some embodiments, the dose-responsive gene features comprise on-target genes that are knocked down in response to each oligonucleotide and off-target genes that are knocked down in response to each oligonucleotide. In some embodiments, the off-target genes comprise sentinel genes. In some embodiments, the off-target genes comprise one or more off-target loci within each off-target gene. In some embodiments, the on-target genes comprise one or more on-target loci within each on-target gene.

In some embodiments, properties of the target genes comprise target loci within the on-target gene. In some embodiments, the dose-responsive gene feature is selected from: RNA-half life, polymerase occupancy, functional genomics features, RNA foundational model target gene loci, toxic off-target effects, and gene expression features. In some embodiments, the dose-responsive gene feature is a gene expression feature comprising on-target genes, off-target genes, single-mismatch genes, or double mismatch genes.

In some embodiments, if the dose-responsive gene feature is or is not determined to be associated with the oligonucleotide sequence, the machine-learned model is configured to further: identify an association between the oligonucleotide sequence or target gene and one or more biomarkers measured from the one or more cells in response to each oligonucleotide administered at different dosages in the set of oligonucleotides.

In some embodiments, the one or more biomarkers is selected from: cytotoxicity, membrane toxicity, immunotoxicity, an effect that inhibits membrane fluidity, a membrane fusion and fission event, and an immune response. In some embodiments, the one or more biomarkers is cellular toxicity. In some embodiments, the one or more biomarkers is liver toxicity.

In some embodiments, the method further comprises: validating a third set of oligonucleotides by synthesizing the third set of oligonucleotides based on the machine-learned model, administering the third set of oligonucleotides to a subject at different dosages, performing the functional genomic analysis operation on DNA or RNA isolated from cells of the subject to quantify gene expression, and measuring a difference between the quantified gene expression and a predicted gene expression produced by the machine-learned model; and modifying the machine-learned model based on the measured difference between the quantified gene expression and a predicted gene expression produced by the machine-learned model.

In some embodiments, the method includes generating a final set of oligonucleotides. using the trained machine-learned model. In some embodiments, the final set of oligonucleotides has one or more of the identified set of characteristics that result in on-target gene knockdown. In some embodiments, the final set of oligonucleotides comprise an IC50 value ranging from 0.1 to 1 μM or 100 nM to 10 μM. In some embodiments, the final set of oligonucleotides target an RNA that has an RNA half-life ranging from 1 minute to 72 hours.

In some embodiments, the kinetic model of dose-response is configured to analyze the time dependence of gene expression in response to the dose of each oligonucleotide of the set of oligonucleotides. In some embodiments, the kinetic model of dose-response is configured to parameterize the mean response of a gene as a function of dose (d) and time (t). In some embodiments, the kinetic model comprises one or more assumptions, wherein the one or more assumptions is selected from: the one or more cells transcribe pre-mRNA at a fixed mean rate β (transcription rate), the pre-mRNA can mature or become bound by the oligonucleotide of the set of oligonucleotide; when an oligonucleotide-pre-mRNA complex is formed, it can be cleaved or become a mature mRNA; the mature RNA decay at a rate of Mδ; the oligonucleotide of the set of oligonucleotides is regulating gene expression through RNAseH mediated knockdown; the oligonucleotide has no effect on the transcription rate β, the mature rate Y, or the mRNA decay rate δ; gene knockdown can saturate to a finite non-zero value; and the maturation rate of state T (regular pre-mRNAs) and state T* (olignucleotide-bound pre-mRNAs) is identical.

In some embodiments, the method is configured to sample probability distributions of the dose-response kinetic model assumptions across at least thousands of genes.

In some embodiments, the noise model is a negative binomial distribution comprising a gene-specific dispersion parameter ϕi, and a mean parameter. In some embodiments, the noise model is scaled by a sample-determined scaling factor sα comprising a total number of non-duplicate reads for a sample, wherein α comprises a sample index. In some embodiments, the noise model is configured to identify biological and technical noise in the functional genomic analysis.

In some embodiments, the Bayesian inference model comprises a Bayesian inference fitter configured to detect dose-response genes and/or quantifying the dose response genes from the functional genomics analysis. In some embodiments, the dose-responsive model comprises fitting dose- and time-dependence of gene expression (e.g., time-dependence in response to dosing of the oligonucleotide).

In some embodiments, the dose-responsive model captures time dependence for genes that show positive dose-response to each oligonucleotide of the set of oligonucleotides. In some embodiments, the dose-responsive model determines a maximum amount of knockdown of gene expression that the oligonucleotide achieves.

In some embodiments, the Bayesian inference model computes whole distribution to construct p-values and credibility intervals that directly quantify how constrained the fit is by the second training set.

In some embodiments, the threshold is a reduction of on-target or off-target gene expression by at least 50%. In some embodiments, the threshold is a reduction of on-target or off-target gene expression by at least 60%. In some embodiments, the threshold is a reduction of on-target or off-target gene expression by at least 70%. In some embodiments, the threshold is a reduction of on-target or off-target gene expression by at least 80%. In some embodiments, the threshold is a reduction of on-target or off-target gene expression by at least 90%.

In some embodiments, the set of oligonucleotides comprises one or more of: a set of aptamers, a set of oligonucleotide-aptamer conjugates, a set of antisense oligonucleotides (ASO), a set of anti-gene oligonucleotides, a set CpG oligonucleotides, a set single-guide RNAs, a set dual-guide RNAs, a set targeter RNAs, a set activator RNAs, a set of LNA oligonucleotides, a set of constrained ethyl (cEt) oligonucleotides, a set of adenosine deaminase acting on RNA (ADAR)-guiding RNA (AD-gRNAs), a set of steric-blocking oligonucleotides (SBOs), a set of antisense oligonucleotides that that recruit endogenously expressed ADARs, a set of antisense oligonucleotides that harness RNase H, a set of intron-targeted ASOs, and a set of exon-targeted ASOs.

### 3. BRIEF DESCRIPTION OF THE SEVERAL VIEWS OF THE DRAWINGS

These and other features, aspects, and advantages of the present invention will become better understood with regard to the following description, and accompanying drawings, where:

The figures are provided in Appendix A. Description of the figures are provided below:

FIG. 1: DoReSeq model diagram. The model describes population of three states T, M, and T* which corresponds to pre-mRNA, mature mRNA, and ASO bound pre-mRNA. Arrows indicate transitions between states and labels over the arrows indicate the transition rates. Arrows pointing to Ø indicate the destruction of the RNA. The arrow starting at β indicates the creation of mRNA at the mean transcription rate β.

FIGS. 2A-2B. DoReSeq dynamics in the regime that mRNA decay rate is much smaller than mRNA maturation rate: δ<<γ. (FIG. 2A) Populations dynamics of pre-mRNA (T(t)), mature mRNA (M(t)), and ASO bound pre-mRNA (T*(t)) as a function of time following ASO injection. Populations are in arbitrary units, time is in units of mRNA maturation rate γ. Inset shows a zoom in on populations T(t) and T*(t) at short times. Observe that following the introduction of the ASO, the populations of T(t) and T*(t) states quickly reach their steady-state values while the population of M(t) state takes much longer time to reach its steady-state value. (FIG. 2B) Mature mRNA response curves obtained with full dynamics and with the approximate δ<<γ dynamics. Inset shows zoom in on the initial dynamics. Observe that the two models predict similar dynamics except for a short-time transient which is absent in the approximate dynamics. [to produce this plot we used α/γ=1, A=5, β/γ=1, δ/γ=0.2, κ/γ=5]

FIGS. 3A-3B. Response as a function of dose at various times after treatment in the regime that mRNA decay rate is much smaller than mRNA maturation rate: δ<<γ. In panel (FIG. 3A) δ/γ=0.2 and in panel (FIG. 3B) δ/γ=0.05. Solid lines correspond to exact solutions Eq. (5) while dashed lines correspond to approximate solutions Eq. (9). Observe that the response saturates with dose and approaches the steady-state solution at long times. Also observe that the discrepancy between exact and approximate solution decreases as the ration δ/γ becomes smaller. [to produce this plot we used α/γ=1, β/γ=1, and κ/γ=5]

FIGS. 4A-4L. Fitting synthetic data with DoReSeq for a strongly dose responsive gene (left column), a weakly dose-responsive gene (middle column) and a dose non-responsive gene (right column). Panels (FIG. 4A-4C): transcripts per million (TPM) vs. dose plots showing the ground truth curves, the synthetic data, and the Maximum A Posteriori (MAP) estimates of TPM vs dose curves (dashed lines) and negative binomial dispersion (green shaded regions. Panels (FIGS. 4D-4I): Markov Chain Monte Carlo samples of the fitting parameters along with global map estimates and ground truth values of the fitting parameters. Panels (FIGS. 4J-4I): marginalized joint probability distributions for the dose-response fitting parameters mKDi and rIC50,i. The global and marginalized MAP estimates of mKDi and rIC50,i are indicated along with the ground truth values. Black contours outline the credibility intervals.

FIGS. 5A-5F. Estimating knockdown at a fixed dose. Panels (FIG. 5A-5C): Knockdown as a function of dose. The gray lines represent a subsample of Monte Carlo data (showing 10,000 out of 160,000 samples). The MAP estimates of mKDi and rIC50,i marginalized over TPMi and ϕi are shown with dashed light blue lines, while the ground truth curves are shown with solid cyan lines. The distribution of knockdown at dose 5 is shown with the solid blue lines, the MAP estimates and 95% credibility intervals derived from these distribution are shown with the purple dots and errorbars. Panel (FIG. 5D-5F): MAP estimates and 95% credibility intervals over 72 replicates of the numerical experiment.

FIGS. 6A-6B. Comparing the dispersion of MAP estimates of mKD and rIC50 (FIG. 6A) to MAP estimates of KD5 (FIG. 6B) for synthetic data generated using three values of ground truth mKD: 0 (blue), 0.5 (orange), and 1 (green).

FIGS. 7A-7E. Effect of varying parameters on fitting synthetic data. Panels (FIG. 7A-7E) show histograms of MAP estimates of Knockdown at Dose=5 obtained on synthetic data as well as the ground truth expectation of the knockdown. Panels (FIG. 7A-7D): we fixed three of the four fit parameters and tuned the value of the remaining fit parameter. The values of the tuned parameter are indicated by tick marks on the horizontal axis (the remaining parameters were fixed to mKD=0, rIC50=2, ϕ=0.05, TPM=50). For each value of the tuned parameter we generated and fitted 288 realizations. We observe that the spread of MAP estimates of knockdown at Dose-5 is weakly dependent on the ground truth values of knockdown mKD and rIC50. On the other hand, the spread of MAP estimates increases with the dispersion parameter ϕ. It also increases as the number of raw reads becomes small, which we control by seeting the ground truth value of the TPM parameter to be small. Panel (FIG. 7E): we fixed mKD=0, rIC50=2, ϕ=0.05, TPM=50 and varied the total number of synthesized samples that we fit. We observe that as the number of samples grows the spread of MAP estimates of knockdown at dose=5 decreases.

FIGS. 8A-8H. Fitting the RNA decay rate parameter δ with time-dependent data. Panel (FIG. 8A-8D): TPM vs dose plots for four different values of δ (as indicated in the column titles). Points represent synthetic data for four different times (as indicated in the legend). Solid lines represent the ground truth TPM vs. dose curves and dashed lines represent the fitted TPM vs. dose curves (with parameters obtained from the global MAP estimate). Panel (FIG. 8E-8H): marginalized probability distributions of δ obtained from the Markov Chain Monte Carlo. Ground truth value of δ is indicated by the dashed black line in each panel. The marginalized MAP estimate and the 95% credibility interval for δ, obtained from the probability distribution, is indicated by the purple dot and bars in each panel.

FIG. 9. Histogram of knockdown at dose=5 p-values computed for experimental data. The histogram shows 203 genes that are strongly knocked down (p-value!0.01), 3573 genes that are weakly knocked down (0.01!P-value!0.99), and 4229 genes that are not knocked-down (0.99! p-value). The vertical axis is split in order to show the peak at high P-values that corresponds to genes that are not knocked down.

FIGS. 10A-10I. Bayesian inference fits of experimentally observed dose-response on a typical strongly dose-responsive gene (left column), weakly dose-responsive gene (middle column) and a dose-non-responsive gene (right column). Panels (FIG. 10A, 10B, 10C) show the experimentally observed dose response for the three genes using the colored points, where color corresponds to the eight plates that were processed to obtain this data. The experimental TPM values are regularized using MAP estimates of TPM on a plate-by-plate basis (see text). The global MAP estimate for dose dependence of TPM and width of negative binomial distributions are indicated with the green dashed line and the green shaded region. Panels (FIG. 10D, 10F,10H) show the histogram for dose-response fit parameters mKDi and rIC50,i. The global and marginalized MAP estimates are indicated by the dots and the 50, 95, and 99% credibility contours by the black lines. Panels (FIG. 10E, 10G,10I) show the histograms for the knockdown at dose=5 along with marginalized MAP estimates and 95% credibility intervals indicated by the purple dots and bars.

FIG. 11 provides cellular toxicity and liver toxicity data of cells that were dosed with oligonucleotides at different dosages. The set of ASOs were categorized based on their cellular and liver toxicities, then profiled their gene expression using Digital Gene Expression (DGE).

FIG. 12 provides RNA expression levels across different dosages for a set of ASOs where RNA expression levels were screened across a dose response. This data was fit through the DoReSeq algorithm to generate a confidence interval for the most likely gene behavior (green shading) for analysis.

FIG. 13 provides gene response data after using the methods of the present disclosure, where gene response to both safe (green) and liver or cytotoxic (red) ASOs (shown on the right) was observed to determine which genes were more likely to be down-regulated by the toxic compounds. Each pixel in this image reflects whether DoReSeq found the gene to be significantly knocked down (black) or not (orange) by a given ASO.

### 4. DEFINITIONS

As used herein and in its conventional sense, “off-target”, refers to a lack of selectivity to a target, which, for example, causes an oligonucleotide to effect a non-target molecule (e.g. non-target gene). In some cases, the non-target molecule is a non-target gene. In some cases, lack of selectivity to a target is caused by the same on-target mechanism for on-target engagement (e.g., RNase H1-mediated mechanism, and the like). In some cases, lack of selectivity to a target is caused by a different mechanism than the intended on-target mechanism for on-target engagement. In some embodiments, the off-target engagement causes the oligonucleotide to perform an effective amount of one or more of: non-target gene expression knock-down, non-target RNA splicing modulatory behavior, non-target gene expression upregulation, non-target gene-editing, non-target RNA-editing, non-target protein specific targeting, non-target receptor specific targeting, non-target enzymatic substrate specific targeting, non-target distribution and uptake into tissues or cells, and non-target interaction with a specific protein or receptor. In some embodiments, off-target engagement is measured by transcriptome-wide gene expression readouts. In some embodiments, off-target engagement of the oligonucleotide to the target is measured by unintended splicing modulation readouts transcriptome-wide. In some embodiments, off-target engagement is measured by biophysical readouts of sequence/edit tolerance of relevant enzymes RNaseH, Ago2 spliceosome factors, and the like.

As used herein, the phrase “on-target” includes on-target engagement of the oligonucleotide to a target molecule. In some embodiments, the on-target engagement causes the oligonucleotide to perform an effective amount of one or more of: gene expression knock-down, RNA splicing modulatory behavior, gene expression upregulation, gene-editing, RNA-editing, interaction with a specific protein or receptor, protein specific targeting, receptor specific targeting, enzymatic substrate specific targeting, and distribution and uptake into tissues or cells.

In some embodiments, the on-target engagement comprises an amount (e.g. %) of gene expression knock-down. In some embodiments, gene expression knock-down can be measured using conventional methods known in the art. In some embodiments, gene expression knock-down is measured by RNase H1 dependent RNA cleavage. In some embodiments, gene expression knock-down is measured by RNA-Induced Silencing Complex (RISC)-dependent

RNA cleavage. In some embodiments, the biophysical effect is RNase H-mediated degradation in the nuclease.

### 5. DETAILED DESCRIPTION

The description of the present disclosure is provided in Appendix A, which is hereby incorporated by reference in its entirety.

An aspect of the present disclosure includes methods for manufacturing oligonucleotide-based medicines.

In some embodiments, the method comprises administering a set of oligonucleotides in vivo or in-vitro.

### 5.1. Oligonucleotides

In some embodiments, the oligonucleotides comprise a set of antisense oligonucleotides (ASO). In some embodiments, the oligonucleotides comprise a set of anti-gene oligonucleotides. In some embodiments, the oligonucleotides comprise a set of CpG oligonucleotides. In some embodiments, the oligonucleotides comprise a set single-guide RNAs. In some embodiments, the oligonucleotides comprise a set dual-guide RNAs. In some embodiments, the oligonucleotides comprise a set targeter RNAs. In some embodiments, the oligonucleotides comprise a set activator RNAs. In some embodiments, the oligonucleotides comprise a set of aptamers. In some embodiments, the oligonucleotides comprise a set of steric-blocking oligonucleotides. In some embodiments, the oligonucleotides comprise a set of ASOs to harness RNase H. In some embodiments, the oligonucleotides comprise a set of tracr RNAs.

In some embodiments, the set of oligonucleotides comprises a set of RNA interference (RNAi)-based oligonucleotides. In some embodiments, the oligonucleotides comprise a set of RNA (ADAR)-guiding RNA (AD-gRNAs). In some embodiments, the oligonucleotides comprise a set of double stranded RNA (dsRNA). In some embodiments, the oligonucleotides comprise a set of CRISPR RNA (crRNA).

### 5.2. In Vitro, In Vivo, and in Silico Experiments

In some embodiments, the method comprises administering a set of oligonucleotides in vivo or in vitro (e.g., experimental).

In some embodiments, the set of oligonucleotides comprises 50 or fewer oligonucleotides, between 50 and 100 oligonucleotides, between 100 and 150 oligonucleotides, between 150 and 200 oligonucleotides, between 200 and 300 oligonucleotides, between 300 and 400 oligonucleotides, between 400 and 500 oligonucleotides, between 500 and 750 oligonucleotides, between 750 and 1000 oligonucleotides, between 1000 and 1500 oligonucleotides, between 1500 and 2000 oligonucleotides, between 2000 and 2500 oligonucleotides, between 2500 to 5000 oligonucleotides, or between 5000 to 10000 oligonucleotides.

In some embodiments, each oligonucleotide of the set of oligonucleotides is administered at a plurality of different dosages. For example, in some embodiments, by administering each oligonucleotide with varying (different) dosages, the method comprises generating a dose-response curve for each oligonucleotide at each dosage tested.

In some embodiments, two or more doses are tested. In some embodiments, three or more doses are tested. In some embodiments, 4 or more, 5 or more, 6 or more, 7 or more, 8 or more, 9 or more, 10 or more, 15 or more, 20 or more, 25 or more, 30 or more, 35 or more, 40 or more, 45 or more, 50 or more, 55 or more, 60 or more, 65 or more, 70 or more, 75 or more, 80 or more, 90 or more, 95 or more, or 100 or more doses are tested. In some embodiments, 100 or more, 120 or more, 125 or more, 150 or more, 200 or more, 225 or more, 250 or more, 275 or more, 300 or more, 325 or more, 350 or more, 375 or more, 400 or more, 425 or more, 450 or more, 475 or more, or 500 or more doses are tested.

In some embodiments, the method comprises administering a set of oligonucleotides in vitro. In some embodiments, the method comprises administering a set of oligonucleotides to one or more cells. In some embodiments, the one or more cells is selected from: eukaryotic single-cell organism, a somatic cell, a germ cell, a stem cell, a plant cell, an algal cell, an animal cell, an invertebrate cell, a vertebrate cell, a fish cell, a frog cell, a bird cell, a mammalian cell, a pig cell, a cow cell, a goat cell, a sheep cell, a rodent cell, a rat cell, a mouse cell, a non-human primate cell, and a human cell.

In some embodiments, the method comprises administering a set of oligonucleotides to a subject in vivo. In some embodiments, in vivo assays are performed on, for example, non-human mammals, mammals, rodents, rats, mice, humans, e.g. rats, mice, pigs, cows, goats, sheep, non-human primates, fish, frogs, vertebrates, and the like.

In some embodiments, the method comprises receiving synthetic data such as synthetic digital gene expression, RNA sequencing (RNA-seq), or tag-based RNA-seq (TAQ-seq) data. In some embodiments, the method comprises receiving NGS data from in vitro or in vivo experiments.

In some embodiments, synthetic data is generated from genomic digital gene expression (DGE) data using a noise model of Equation (14).

In some embodiments, each oligonucleotide within a set of oligonucleotides is administered at a different dosage. These experiments are oligonucleotide-based medicine dose-response experiments. Thus, by administering each oligonucleotide at different dosages, the method can study a dose-response relationship between quantified and predicted on-target and off-target gene effects in response to administering the oligonucleotides.

In some embodiments, the method characterizes the effects the level of gene expression dependent upon the concentration of an oligonucleotide as detected e.g., by next generation sequencing (NGS) at every dose-point. In some embodiments, the methods of the present disclosure identify which genes are knocked down (whether on-target or off-target) as well as quantify both the level of knockdown and the confidence in the model in such knockdown calls.

In some embodiments, the method comprises measuring the “efficiency” of non-homologous end joining (NHEJ) and/or homology directed repair (HDR) and/or other hybridization parameters after administration of the oligonucleotide, which can be calculated by any convenient method. For example, in some cases, efficiency can be expressed in terms of percentage of successful HDR. For example, a restriction digest assay (e.g., using a restriction enzyme such as HindIII) can be used to generate cleavage products and the ratio of products to substrate can be used to calculate the percentage. For example, a restriction enzyme can be used that directly cleaves DNA containing a newly integrated restriction sequence as the result of successful HDR. More cleaved substrate indicates a greater percent HDR (a greater efficiency of HDR). As an illustrative example, a fraction (percentage) of HDR can be calculated using the following equation [(cleavage products)/(substrate plus cleavage products)] (e.g., b+c/a+b+c), where “a” is the band intensity of DNA substrate and “b” and “c” are the cleavage products.

In some embodiments, efficiency can be expressed in terms of percentage of successful NHEJ. For example, a T7 endonuclease I assay can be used to generate cleavage products and the ratio of products to substrate can be used to calculate the percentage NHEJ. T7 endonuclease I cleaves mismatched heteroduplex DNA which arises from hybridization of wild-type and mutant DNA strands (NHEJ generates small random insertions or deletions (indels) at the site of the original break). More cleavage indicates a greater percent NHEJ (a greater efficiency of NHEJ). As an illustrative example, a fraction (percentage) of NHEJ can be calculated using the following equation: (1−(1−(b+c/a+b+c))1/2)×100, where “a” is the band intensity of DNA substrate and “b” and “c” are the cleavage products (see e.g., Ran et. al., Cell. 2013 Sep. 12; 154(6): 1380-9). This formula is used (instead of the formula used for HDR, see above) because upon re-annealing, one duplex of mutant DNA can produce two duplexes of mutant: wild-type hybrid, doubling the actual NHEJ frequency.

In some embodiments, the method comprises measuring the ability of each oligonucleotide to hybridize to the target gene. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of the melting temperature (Tm) for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived (see Sambrook et al., supra, 9.50-9.51). For hybridizations with shorter nucleic acids, i.e., oligonucleotides, the position of mismatches becomes more important, and the length of the oligonucleotide determines its specificity (see Sambrook et al., supra, 11.7-11.8). Typically, the length for a hybridizable nucleic acid is at least about 10 nucleotides. Illustrative minimum lengths for a hybridizable nucleic acid are: at least about 15 nucleotides; at least about 20 nucleotides; and at least about 30 nucleotides. Furthermore, the skilled artisan will recognize that the temperature and wash solution salt concentration may be adjusted as necessary according to factors such as length of the probe.

### 5.3. Functional Genomic Analysis

In some embodiments, the method further comprises performing a functional genomic analysis operation on the one or more cells to quantify gene expression for each oligonucleotide dosage.

In some embodiments, functional genomics includes, but is not limited to sequence-based assays, such as NGS assays, DNA/RNA sequencing assay-based readouts, and/or protein-level readouts, such as proteomics, western blot, and ELISA assays for corresponding RNAs.

In some embodiments, a functional genomic analysis operation analyzes gene expression, RNA transcripts, protein interactions, epigenetic changes, and a combination thereof. In some embodiments, the functional genomic analysis operation is a gene expression analysis that quantifies and compares gene expression levels. In some embodiments, the functional genomic analysis operation analyzes of all RNA transcripts or transcriptome. In some embodiments, the functional genomic analysis operation analyzes protein expression and function. In some embodiments, the functional genomic analysis operation quantifies the entire RNA content, including splicing variants. In some embodiments, the functional genomic analysis provides targeted expression profiling.

In some embodiments, performing a functional genomic analysis comprises performing a functional genomic analysis operation comprises performing NGS.

In some embodiments, performing a functional genomic analysis comprises performing a functional genomic analysis operation comprises performing DGE. In some embodiments, the the output of DGE experiments can include a timepoint readout. In some embodiments, performing a functional genomic analysis comprises performing a functional genomic analysis operation comprises performing RNA sequencing (RNA-seq). In some embodiments, performing a functional genomic analysis comprises performing a functional genomic analysis operation comprises performing tag-based RNA sequencing (TAQ-seq).

### 5.4. Dose-Responsive Model

In some embodiments, the method comprises creating a training data set by fitting a dose-responsive model to a dose-dependent response of each oligonucleotide on gene expression.

In some embodiments, the dose-dependent response of each oligonucleotide on gene expression was determined by the functional genomic analysis.

In some embodiments, the dose-responsive model is configured to identify and characterize dose-responsive gene features dependent upon the dosage of each oligonucleotide of the set of oligonucleotides.

In some embodiments, the dose-responsive model comprises fitting dose- and time-dependence of gene expression (e.g., time-dependence in response to dosing of the oligonucleotide).

In some embodiments, the dose-responsive model captures time dependence for genes that show positive dose-response to each oligonucleotide of the set of oligonucleotides.

In some embodiments, the dose-responsive model determines a maximum amount of knockdown of gene expression that the oligonucleotide achieves.

### 5.4.1. Kinetic Model of Dose-Response

In some embodiments, the dose-responsive model comprises a kinetic model of dose-response.

In some embodiments, the kinetic model of dose-response is configured to analyze the time dependence of gene expression in response to the dose of each oligonucleotide of the set of oligonucleotides.

In some embodiments, the kinetic model of dose-response is configured to parameterize the mean response of a gene as a function of dose (d) and time (t).

In some embodiments, the kinetic model comprises one or more assumptions, wherein the one or more assumptions is selected from: the one or more cells transcribe pre-mRNA at a fixed mean rate β (transcription rate), the pre-mRNA can mature or become bound by the oligonucleotide of the set of oligonucleotide; one an oligonucleotide-pre-mRNA complex is formed, it can be cleaved or become a mature mRNA; the mature RNA decay at a rate of Mδ; the oligonucleotide of the set of oligonucleotides is regulating gene expression through RNAseH mediated knockdown; the oligonucleotide has no effect on the transcription rate β, the mature rate Y, or the mRNA decay rate δ; gene knockdown can saturate to a finite non-zero value; and the maturation rate of state T (regular pre-mRNAs) and state T* (olignucleotide-bound pre-mRNAs) is identical. In some embodiments, the kinetic model comprises two or more, three or more, four or more, or five or more assumptions, wherein the two or more, three or more, four or more, or five or more assumptions are selected from: the one or more cells transcribe pre-mRNA at a fixed mean rate β (transcription rate), the pre-mRNA can mature or become bound by the oligonucleotide of the set of oligonucleotide; one an oligonucleotide-pre-mRNA complex is formed, it can be cleaved or become a mature mRNA; the mature RNA decay at a rate of Mδ; the oligonucleotide of the set of oligonucleotides is regulating gene expression through RNAseH mediated knockdown; the oligonucleotide has no effect on the transcription rate β, the mature rate Y, or the mRNA decay rate δ; gene knockdown can saturate to a finite non-zero value; and the maturation rate of state T (regular pre-mRNAs) and state T* (olignucleotide-bound pre-mRNAs) is identical.

In some embodiments, the kinetic model comprises the following assumptions: the one or more cells transcribe pre-mRNA at a fixed mean rate β (transcription rate), the pre-mRNA can mature or become bound by the oligonucleotide of the set of oligonucleotide; one an oligonucleotide-pre-mRNA complex is formed, it can be cleaved or become a mature mRNA; the mature RNA decay at a rate of Mδ; the oligonucleotide of the set of oligonucleotides is regulating gene expression through RNAseH mediated knockdown; the oligonucleotide has no effect on the transcription rate β, the mature rate Y, or the mRNA decay rate δ; gene knockdown can saturate to a finite non-zero value; and the maturation rate of state T (regular pre-mRNAs) and state T* (olignucleotide-bound pre-mRNAs) is identical.

In some embodiments, the results of the kinetic model of dose-response is that knockdown can saturate to a finite, non-zero value, e.g., even at very high oligonucleotide concentrations. In some embodiments, the kinetic model captures a biological mechanism: a mechanism where the target-site may not be available for productive cleavage that leads to gene knockdown. In some embodiments, the kinetic model captures a second biological mechanism: a the maturation rate of state T (regular pre-mRNAs) and state T* (oligonucleotide-bound pre-mRNAs) is identical. In some embodiments, where the oligonucleotide is an antisense oligonucleotide (ASO), ASOS act co-transcriptionally, and this could be because of RNA regulatory processes, such as splicing removing, the target-site into intronic lariats, RNA secondary structure, RNA-binding protein occupancy (RBPs), regulatory RNA occupancy reducing accessibility to target-site, and therefore, to ASO cleavage. In some embodiments, the ASO: RNA bound state is a local stretch of 10-20 base pairs (Bps) long of the pre-mRNA, and the fate of the pre-mRNA without RNaseH activity is typically determined at a significantly longer length scale.

In some embodiments, the kinetic model dose-response makes the assumption that the RNAs are transcribed at a steady state.

The method of any one of claims 1-20, wherein the method is configured to sample probability distributions of the dose-response kinetic model assumptions across at least thousands of genes.

In some embodiments, the kinetic model includes first-order interactions for RNaseH-mediated knockdown.

The processes described above result in the kinetic equations described herein. The kinetic equations of the model and their properties are described below.

$$\frac{d}{dt} [ \begin{matrix}T \\ M \\ {T}^{*}\end{matrix} ] = [ \begin{matrix}- ( γ + α A ) & 0 & 0 \\ γ & - δ & γ \\ α A & 0 & - ( κ + γ )\end{matrix} ] [ \begin{matrix}T \\ M \\ {T}^{*}\end{matrix} ] + [ \begin{matrix}β \\ 0 \\ 0\end{matrix} ] \qquad ( Equation ( 1 ) )$$

In some embodiments, to analyze the implications of these rate equations, the steady state populations were found in the absence of oligonucleotide treatment and then these steady-state populations were used as the initial condition for time evolution following oligonucleotide treatment.

To find the initial, steady-state populations T₀, M₀, and T₀*, the LHS was set of eq. (1) to zero and also set the oligonucleotide concentration A=0 to obtain:

$${T}_{0} = β / γ {M}_{0} = β / δ {T}_{0}^{*} = 0 , \qquad ( Equation ( 2 ) )$$

$$T ( t ) = \frac{β}{A α + γ} ( 1 + \frac{A α {e}^{- t ( A α + γ )}}{γ} ) , \qquad ( Equation ( 3 ) )$$

$$M ( t ) = \frac{A αβκ {e}^{- t ( A a + γ )}}{( A α + γ ) ( A α + γ - δ ) ( A α - κ )} - \frac{A αβκ {e}^{- t ( γ + κ )}}{( A α - κ ) ( γ + κ ) ( γ - δ + κ )} + \frac{A αβκ {e}^{- t δ}}{δ ( A α + γ - δ ) ( γ - δ + κ )} + \frac{βγ ( A α + γ + κ )}{δ ( A α + γ ) ( γ + κ )} , \qquad ( Equation ( 4 ) )$$

$$T * ( t ) = A αβ ( \frac{κ {e}^{- t ( γ + κ )}}{γ ( A α - κ ) ( γ + κ )} - \frac{A α {e}^{- t ( A a + γ )}}{γ ( A α + γ ) ( A α - κ )} + \frac{1}{γ ( A α + γ ) ( γ + κ )} ) . \qquad ( Equation ( 5 ) )$$

Now, the focus included population of mature mRNAs, as that is the quantity that is being measured experimentally. Taking the long-time limit, the steady state population was found as shown below:

$$M ( t \to ∞ ) = \frac{β}{δ} ( 1 - \frac{κ}{γ + κ} \frac{A α}{A α + γ} ) . \qquad ( Equation ( 6 ) )$$

From this expression, the maximum knockdown (saturation effect) can be read off at high oligonucleotide concentration to be:

$$m K D = \frac{M ( t \to ∞ , A \to ∞ )}{{M}_{0}} = \frac{γ}{γ + κ} . \qquad ( Equation ( 7 ) )$$

This expression highlights that the maximum knockdown is controlled by the competition of mRNA maturation rate γ and the oligonucleotide guided cleavage rate κ. For fitting experimental observations, it was convenient to re-parameterize the response using mKD and rIC50=γ/α:

$$R ( t \to ∞ , A ) = \frac{M ( t \to inf )}{{M}_{0}} = 1 - \frac{A ( 1 - m K D )}{A + {rIC}_{50}} \qquad ( Equation ( 8 ) )$$

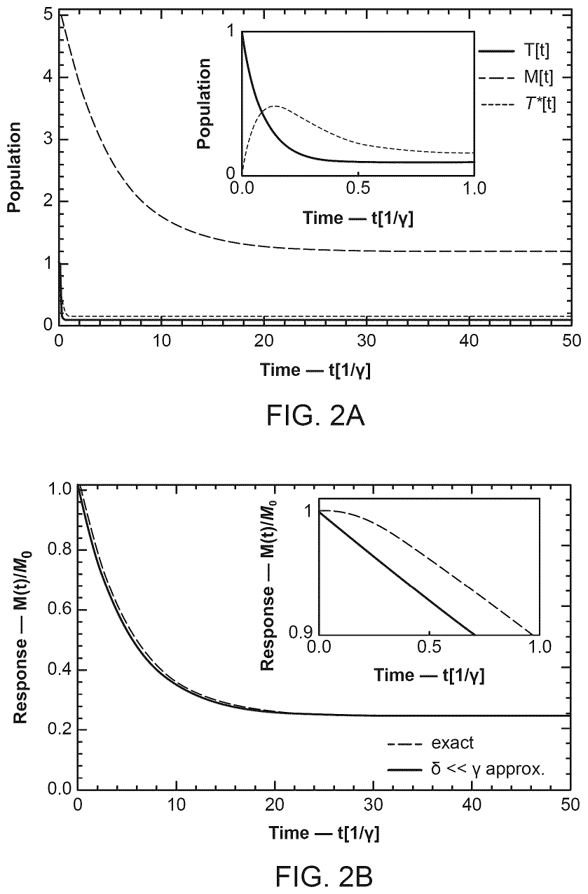

Next, the model explored how M(t) approaches the steady state. The expression for the time dependence of M(t) in equation Eq. (5) is rather complicated, involving four different terms, one static and three exponential decays controlled by three different rates: Aα+γ, γ+κ, and δ. To gain intuition, the constituent rates Aα, γ, κ, and δ were first looked at. In order for knockdown to occur κ, Aα≥γ, otherwise there will not be appreciable flow T→T*→cleavage. On the other hand, it was expected to be in the regime δ<γ, which is usually appropriate for genes in eukaryotic cells in which mRNA maturation time is much shorter than mRNA half-life. Hence it was expected to be a short transient associated with the fast rates Aα+γ and γ+κ in which the populations T(t) and T*(t) reach their steady-state values, followed by a long decay associated with rate δ over which M(t) reaches its steady-state value. Which is indeed what was observed as shown in FIG. 2, as for the case δ=0.27 γ.

Next the following simplifying assumption was made which was that δ<<γ, i.e. the initial transient was ignored in which the populations T(t) and T*(t) reach their steady-state values. Under this assumption, the two fast decaying terms were dropped and simplified the denominator of the remaining term of M(t) in Eq. (5) to obtain the response function:

$$R ( t , A ) = \frac{M ( t )}{{M}_{o}} \approx 1 - \frac{A ( 1 - m K D )}{A + {rIC}_{50}} ( 1 - {e}^{- t δ} ) , \qquad ( Equation ( 9 ) )$$

where the same parametrization was used in terms of mKD and rIC50 as in Eq. (8). In FIG. 2 the response function obtained was compared from the exact time evolution Eq. (5) and from the approximate time evolution Eq. (9). It was observed that the two curves were almost identical, except for the small initial transient that can be seen in the zoomed view plotted in the inset.

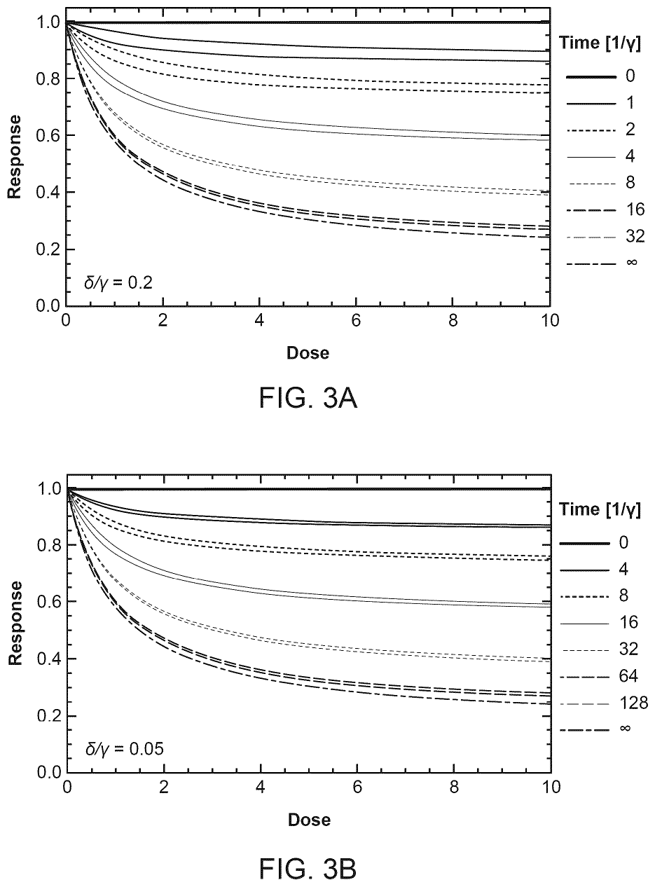

In FIG. 3, plot response curves were plotted as a function of dose for various times using both the exact solution of Eq. (5) as well as the approximate solution Eq. (9). It was observed that if time was fixed, the response increases with dose saturating at high dose. At the same time, it was observed that as time increases so does the response until the steady state is reached at long times. Finally, it was observed that for δ≤0.2 γ, exact and approximate solutions of the kinetic model are almost identical. Eq. (9) is a simple and convenient expression for parameterizing the response of gene expression to oligonucleotide treatment.

### 5.4.2. Noise Model of Gene Expression

In some embodiments, the dose-responsive model further comprises a noise model of gene expression. In some embodiments, the noise model of gene expression is used to calibrate the tissue, cell-type, etc. specific variation/noise of gene expression and confidence (signal-to-noise) of gene knockdown at every dose.

In some embodiments, the noise model is a negative binomial distribution comprising a gene-specific dispersion parameter ϕi, and a mean parameter.

In some embodiments, the noise model is scaled by a sample-determined scaling factor sa comprising a total number of non-duplicate reads for a sample, wherein α comprises a sample index. In some embodiments, this is a batch and tissue-specific scaling to take care of differences in read depth, bulk-RNA quantity & quantity and determined by experiments.

In some embodiments, the noise model is configured to identify biological and technical noise in the functional genomic analysis operation.

In some embodiments, n order to connect the dose response model to the noise in gene expression, the following expression was introduced for the expected mean number of counts of gene i in sample α:

$$μ = [ \frac{{s}_{α} T P {M}_{i}}{1 {0}^{6}} ] [ 1 - \frac{{A}_{α} ( 1 - m K {D}_{i} )}{{A}_{α} + {rIC}_{50 , i}} ( 1 - {e}^{- {t}_{α} {δ}_{i}} ) ] , \qquad ( Equation ( 10 ) )$$

where sα is the scaling parameter for sample α; Aα is the ASO dose in sample α; tα is the time at which sample α was read out; TPMᵢ is the zero-dose transcripts per million value for gene i; mKDᵢ, rIC50, i, and δᵢ are the gene-specific DoReSeq parameters.

In some embodiments, in this analysis s_{α}=N_{α} to be the total number of non-duplicate reads of all genes in sample α. In some embodiments, the term in the first bracket on the RHS of Eq. (10) is the expected number of counts for gene i at zero dose, while the term in the second bracket is the fractional decrease in the counts induced by the treatment. In the following, sα, Aα, and tα will be treated a confounding/independent variables, and TPMi, mKDi, rIC50, i, and δᵢ as fit parameters.

In some embodiments, the Negative Binomial distribution is used to described the noise in the NGS data, where the noise can be both biological or technical in nature. To simplify notation, Negative Binomial distribution PNB(x|μ, r) was parameterized using the mean, u, and the dispersion parameter, ϕ. This parametrization is connected to the more standard parametrization in terms of p and r via

$$p = \frac{1}{1 + μ ϕ} r = \frac{1}{ϕ} . \qquad ( Equation ( 11 ) )$$

where x_{α,i} is the number of reads of gene i in sample α and ϕi is the gene-specific dispersion parameter. In other words, Eq. (14) says that that the distribution of x_{α,i} is the negative binomial distribution with mean set by the expected number of reads from Eq. (10) and width set by the gene specific dispersion parameter ϕi. Further, in order to determine the expected number of reads, its necessary to have the values of the confounding/independent variables s_{α}, A_{α}, and t_{α} for sample α which are derived from the experimental data as well as the values of the fitting parameters TPMi, mKDi, rIC50,i, and δi.

In some embodiments, for the case where all measurements were carried out at the same time after treatment, the fitting parameter δi can be dropped and the expression (1−e^{-tαδi}) set to unity.

### 5.4.3. Bayesian Inference Model

In some embodiments, the dose-responsive model further comprises a Bayesian inference model.

In some embodiments, the Bayesian inference model can include dose-responsive gene features. In some embodiments, the gene features that are observed and discovered from experimentation can include, but are not limited to: RNA-half life, polymerase occupancy, functional genomic features, RNA foundation model target gene loci, and gene expression features.

In some embodiments, the Bayesian inference model comprises a Bayesian inference fitter configured to detect dose-response genes (or dose-responsive gene features) and/or quantifying the dose response genes from the functional genomics analysis.

In some embodiments, the Bayesian inference model computes whole distribution to construct p-values and credibility intervals that directly quantify how constrained the fit is by the second training set.

In some embodiments, wherein fitting a dose-responsive model comprises fitting dose response using Bayesian inference. In some embodiments, by fitting dose response using Bayesian inference, the method is configured to find the probability distribution of the fit parameters:

$$= \{ T P {M}_{i} , m K {D}_{i} , {rIC}_{50 , i} , {δ}_{i} , {ϕ}_{i} \} : \qquad ( Equation ( 15 ) )$$

$$P ( {ℱ}_{i} \vert \{ {x}_{α , i} \} , \{ {𝒞}_{α} \} ) = \frac{P ( \{ {x}_{α , i} \} \vert {ℱ}_{i} , \{ {𝒞}_{α} \} ) P ( {ℱ}_{i} )}{\int d {ℱ}_{i} P ( \{ {x}_{α , i} \} \vert {ℱ}_{i} , \{ {𝒞}_{α} \} )} ,$$

where {Cα}={s_{α}, dose_{α}, t_{α}} is the set of confounding/independent variables. Eq. (14) was used to define the likelihood

$$P ( \{ {x}_{α , i} \} \vert {ℱ}_{i} , \{ {𝒞}_{a} \} ) = {\prod }_{i} {P}_{NB} ( {x}_{α , i} \vert {ℱ}_{i} , {𝒞}_{α} ) . \qquad ( Equation ( 16 ) )$$

With the parametrization, the mean and variance of the negative binomial distribution are:

$${\sum }_{x = 0}^{∞} x {P}_{NB} ( x \vert μ , r ) = μ , \qquad ( Equation ( 12 ) )$$

$${\sum }_{x = 0}^{∞} {( x - μ )}^{2} {P}_{NB} ( x \vert μ , r ) = μ + {μ}^{2} ϕ . \qquad ( Equation ( 13 ) )$$

In some embodiments, fitting the dose response using Bayesian inference further comprises introducing a model for noise in the number of reads. The following equation is provided below:

$$P ( {x}_{α , i} \vert {s}_{α} , {A}_{α} , {t}_{α} , {TPM}_{i} , {mKD}_{i} , {rIC}_{50 , i} , {δ}_{i} , {ϕ}_{i} = {P}_{NB} ( {x}_{α , i} \vert [ \frac{{s}_{α} {TPM}_{i}}{1 {0}^{6}} ] [ 1 - \frac{{A}_{α} ( 1 - {mKD}_{i} )}{{A}_{α} + {rIC}_{50 , i}} ( 1 - {e}^{- {t}_{α} {δ}_{i}} ) ] , {ϕ}_{i} ) , \qquad ( Equation ( 14 ) )$$

In some embodiments, for the prior P( [symbol] ), box distributions are used with reasonable range for each of the fit parameters:

$$0.001 < {TPM}_{i} < 50000 , \qquad ( Equation ( 17 ) )$$

$$0.001 < {mKD}_{i} < 1 , \qquad ( Equation ( 18 ) )$$

$$0.001 < {rIC}_{50 , i} < 15 , \qquad ( Equation ( 19 ) ) ,$$

$$0.001 < {ϕ}_{i} < 10 , \qquad ( Equation ( 20 ) )$$

$$0.04 < {δ}_{i} < 1 [ 1 / hours ] , \qquad ( Equation ( 21 ) )$$

where δi is the only quantity with units and it was measured in inverse hours as indicated.

In some embodiments, in order to efficiently explore the five dimensional space of fit parameters (four dimensional in case time-dependent data is not available), the method comprises using a Markov chain Monte Carlo (MCMC) to sample the numerator of the RHS of Eq. (15).

In some embodiments, the output of MCMC is a list of samples: 5-dimensional (4-dimensional if there is no time-dependence) vectors that define a set of models. The vectors sample the probability density defined by Eq. (15)—there are more vectors clustered in the regions where P ( [symbol] i|{xα,i}, {Cα}) is large and fewer in the regions where it is small. In some embodiments, the sampling can be performed using the emcee python package.

To turn the list of vectors sampled by MCMC into a probability density, the method comprises generating a normalized histogram. In some embodiments, the method comprises constructing three types of histograms: (1) histograms of the parameters that specify the noise distribution FDS={TPMi, ϕi}; (2) histograms of the dose response parameters [symbol] _{DR}=data or [symbol] _{DR}={mKDi, rIC50,i} for the case there is only data at a single time-point; (3) histograms of the observable KD_{dose,time}, the knockdown at fixed dose and time.

The histograms are used to obtain the maximum a posteriori (MAP) estimates of the fit parameters, credibility regions containing 50%, 95%, and 99% of the probability, and p-values for KD_{dose, time} to be above a threshold. From the credibility contours, “error bars” are also obtained which correspond to the extent of the credibility contour in the cardinal directions. The three analysis that are performed are now described herein.

In some embodiments, the method comprises analyzing the noise distribution histograms, which are configured to provide an estimate of the fit parameters [symbol] DS={TPMi, ϕi}, which are needed for identifying poorly-behaved genes, i.e. genes that have a low number of reads (e.g. TPMi<10) or large variability (e.g. ϕi>0.2).

In some embodiments, the method comprises analyzing noise distribution histograms interpreting dose-response analysis. In some embodiments, the noise distribution histograms are configured to provide a basis for understanding why certain genes have larger credibility intervals than others when fitting dose response.

In some embodiments, the dose-response histograms are configured to provide the data for quantifying the dose-response parameters.

Below, it is shown that the probability density function P(F) is not separable, and therefore it is important to analyze it as a joint probability distribution.

In some embodiments, the method comprises analyzing fixed-dose, fixed-time response histograms that combine the three dose-response fit parameters [symbol] DR={mKDi, rIC50,i, δi} into a single, biologically significant observable KDdose,time. KDdose,time, for predicting the outcome of a particular treatment: e.g. how much knockdown can one expect at a fixed treatment dose. In some embodiments, the KDdose,time is an observable that allows for identifying strongly-, weakly-, and none-dose-responsive genes, e.g. using a p-value.

In some embodiments, the method further comprises randomly sampling the negative binomial distributions. In order to visualize the simulated data, the synthetic values of raw number of reads xαi was converted to synthetic TPM (transcripts per million) using:

$${TPM}_{a , i} = [ \frac{{s}_{α} {x}_{α , i}}{{10}^{6}} ] ,$$

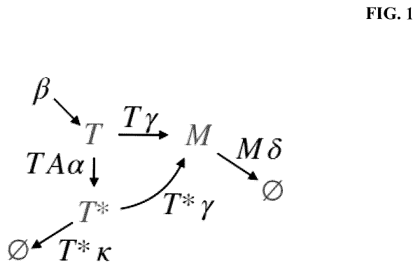

and plotted the results in FIG. 1(a-c) using blue circles. The ground truth was also plotted.

In some embodiments where the method receives a synthetic dataset or the synthetic data was generated using the noise model, the method comprises using the Bayesian inference model to analyze the synthetic dataset.

In some embodiments, where the method comprises administering a set of oligonucleotides in vivo or in vitro (e.g., experimental) and generating experimental data by performing a functional genomic analysis operation, the method comprises applying the machinery of the dose-responsive model to analyze an experimental functional genomic analysis operation dataset. In some embodiments, the dose-responsive model identifies dose-responsive genes and quantifies their response.

In order to remove unwanted variance from plate to plate variations, when applying the Bayesian machinery to experimental data, a separate and independent TPM parameter was used for each plate.

Specifically, Eq. (14) is replaced with

$$P ( {x}_{α , i} \vert {s}_{α} , {A}_{α} , {t}_{α} , \{ TP {M}_{i , p ( α )} \} , mK {D}_{i} , {rIC}_{50 , i} , {δ}_{i} , {ϕ}_{i} ) = {P}_{NB} ( {x}_{α , i} \vert μ , {ϕ}_{i} ) , \qquad Equation ( 28 ) ,$$

$$μ = [ \frac{{s}_{α} {TPM}_{i , p ( α )}}{{10}^{6}} ] [ 1 - \frac{{A}_{α} ( 1 - {mKD}_{i} )}{{A}_{α} + {rIC}_{50 , i}} ( 1 - {e}^{- {t}_{α} {δ}_{i}} ) ] ,$$

where {TPMi,p(α)} is a collection of TPM values associated with gene i, with one value assigned to each plate p(α). In this case there were 8 plates and hence 8 values of {TPMi,p(α)} were fitted for each gene. In order to compare experimentally obtained TPM values across different plates, a normalization scheme was introduced where we normalize the experimentally observed TPM value, on a plate-by-plate basis, to the mean of the MAP estimates of TPM over the plates:

$${TPM}_{norm , i , p} = {TPM}_{obs , i , p} \frac{{〈 {TPM}_{MAP , i , p} 〉}_{p}}{{TPM}_{MAP , i , p}} . \qquad Equation ( 29 ) .$$

In some embodiments, the method comprises implementing the mixed probability distribution method for attenuating outliers. In this method, the Negative Binomial distribution is supplemented for inliers with a much wider Negative Binomial distribution for outliers.

In order to identify dose-responsive genes, we computed the p-value of the knockdown at dose=5 for each of the 8005 genes. The p-value was evaluated using Eq. (26) with threshold set to θ=0.8. The histogram of p-values, see FIG. 9, appears to be bimodal with a peak at small p-values that corresponds to strongly dose-responsive genes and another peak at large p-values that corresponds to the dose-non-responsive genes.

Next, in order to better understand the characteristics of the genes in the two peaks of the p-value distribution, as well as genes in between, the method comprises choosing three representative genes with TPM ˜50, one strongly dose responsive gene (p-value=0), one weakly responsive gene (p-value=0.123) and one dose non-responsive gene (pvalue=1).

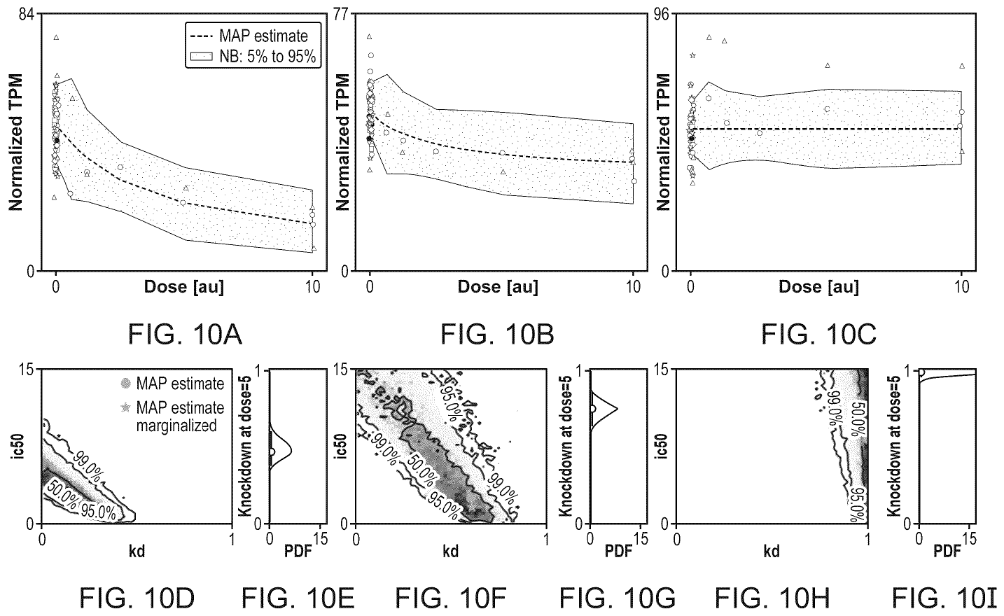

To visualize the dose response, the method comprises plotting the normalized experimental TPM values (see Eq. 24) as a function of dose in FIG. 10(a-c). Superimposed on the experimental points are MAP estimates of TPM as a function of dose (Eq. 24 obtained using the plate-averaged value of TPM: TPMi→ [symbol] TPM_{MAP,i,p} [symbol] ₚ) as well as the MAP estimate of the dispersion encoded as the 5% and 95% quantiles of the negative binomial distributions

To understand the quality of the fit, the method comprises analyzing the probability distributions for the knockdown parameters.

### 5.5. Dose-Responsive Gene Features

In some embodiments, the method comprises creating a training set by fitting a dose-responsive model to a dose-dependent response of each oligonucleotide on gene expression, wherein the dose-responsive model is configured to identify and characterize dose-responsive gene features dependent upon the dosage of each oligonucleotide of the set of oligonucleotides.

In some embodiments, the dose-responsive gene feature is on-target genes that are knocked down in response to each oligonucleotide at a specific dose.

In some embodiments, the dose-responsive gene feature is off-target genes that are knocked down in response to each oligonucleotide at a specific dose.

In some embodiments, the off-target genes comprise sentinel genes.

In some embodiments, the off-target genes comprise one or more off-target loci within each off-target gene.

In some embodiments, the on-target genes comprise one or more on-target loci within each on-target gene. In some embodiments, properties of the target genes comprise target loci within the on-target gene.

In some embodiments, the dose-responsive gene feature is selected from one or more of: RNA-half life, polymerase occupancy, functional genomics features, RNA foundational model target gene loci, toxic off-target effects, and gene expression features.

In some embodiments, the dose-responsive gene feature is a gene expression feature comprising on-target genes, off-target genes, single-mismatch genes, or double mismatch genes.

In some embodiments, the gene feature is on-target engagement. In some embodiments, the on-target engagement comprises an amount of splicing modulatory behavior. RNA mis-splicing causes a large array of human diseases due to hereditary and somatic mutations. In some embodiments, the biophysical effect comprises recognition of specific RNA splicing regulatory elements to modulate splicing.

In some embodiments, the on-target engagement comprises the amount (e.g. %) of gene expression up-regulation. In some embodiments, on-target engagement is the amount of gene expression upregulation determined by stabilization of RNA through reduction of endogenous RNA degradation pathways, such as, but not limited to, miRNA directed RISC cleavage, protracted maintenance of polyA tails, and stabilization of RNA structures, including polysome formation. In some embodiments, on-target engagement is the amount of gene expression upregulation determined by enhanced translation through blockage of non-productive uORFs. In some embodiments, on-target engagement is the amount of gene expression upregulation determined by OBM-directed recruitment of nuclear factors. In some embodiments, OBM directed recruitment of nuclear factors is determined by directly binding DNA. In some embodiments, OBM directed recruitment of nuclear factors is determined by indirectly binding DNA through interactions that orchestrate productive chromatin organization or dynamics.

In some embodiments, on-target engagement comprises an amount of gene-editing. In some embodiments, gene-editing is achieved by CRISPR (Clustered Regularly Interspaced Short Palindromic Repeats)/Cas and CRISPR-like enzymatic processes. In some embodiments, gene editing is achieved by engagement with other endogenous DNA repair and editing mechanisms, such as, but not limited to, zinc-finger nucleases (ZFNs) and transcription activator-like effector nucleases (TALENs). Using a guide RNA, Cas endonuclease (e.g., Cas9) can modify (e.g. cleave) double-stranded DNA at any site, defined by the guide RNA sequence, and including a protospacer-adjacent (PAM) motif. A Cas/guide RNA complex (i.e., a Cas targeting complex) constitutes a simple and versatile RNA-directed system for modifying target DNA, or modifying proteins associated with target DNA, in any desired cell or organism. Additionally, a Cas targeting complex having a mutated Cas9 protein with reduced or removed nuclease activity can still bind to target DNA.

In some embodiments, the dose-responsive gene feature is gene an amount of gene expression. In some embodiments, gene expression is calculated as transcripts per million (TPM), reads per kilobase of transcript per million (RPKM), fragment per kilobase of transcript per million (FPKM). In some embodiments, gene expression is calculated as differential expression (log 2FC, p-value).

In some embodiments, the dose-responsive gene feature includes genomic coordinates such as chromosomal location. In some embodiments, the dose-responsive gene feature comprises promoter/enhancer activity such as regions driving expression of the gene. In some embodiments, the dose-responsive gene feature comprises epigenetic markers.

In some embodiments, dose-responsive gene features comprise the number and type of mismatch genes. In some embodiments, the dose-responsive gene features comprise off-target expression change. In some embodiments, the dose-responsive gene features comprise predicted binding sites.

In some embodiments, the dose-responsive gene features comprise RNA half-life. In some embodiments, the dose-responsive gene features comprise degradation rate. In some embodiments, the dose-responsive gene features comprise step loop structures or UTR elements that effect decay. In some embodiments, the dose-responsive gene feature comprises RNA-binding proteins (RBPs).

In some embodiments, the dose-responsive gene features comprise single mismatch genes (e.g., genes with one nucleotide mismatch of the targeting sequence). In some embodiments, the dose-responsive gene feature comprises double mismatch genes (e.g., genes with two mismatches that are possibly subject to off-target effects).

In some embodiments, the dose-responsive gene feature comprises seed region matches. In some embodiments, the dose-responsive gene feature comprises PAM proximity.

In some embodiments, if the dose-responsive gene feature is or is not determined to be associated with the oligonucleotide sequence, the machine-learned model is configured to further: identify an association between the oligonucleotide sequence or target gene and one or more biomarkers measured from the one or more cells in response to each oligonucleotide administered at different dosages in the set of oligonucleotides.

In some embodiments, the one or more biomarkers is selected from: cytotoxicity, membrane toxicity, immunotoxicity, an effect that inhibits membrane fluidity, a membrane fusion and fission event, and an immune response. In some embodiments, the one or more biomarkers comprise an oligonucleotide dose-dependent toxicity response.

### 5.6. Training a Machine Learned Model

In some embodiments, the method comprises training a machine-learned model using the training set created by fitting the dose-responsive model to the dose-dependent response of each oligonucleotide on gene expression.

In some embodiments, the machine learned model is configured to determine whether the dose-responsive gene feature is or is not associated with the oligonucleotide sequence.

In some embodiments, the machine learned model is configured to determine whether the dose-responsive gene feature is associated with an on-target or off-target effect.

In some embodiments, if the dose-responsive gene feature is determined to be associated with the oligonucleotide sequence, the machine-learned model is configured to further: identify properties of the oligonucleotide sequence or target genes that result in on-target gene knockdown above a threshold.

In some embodiments, if the dose-responsive gene feature is determined to be associated with the oligonucleotide sequence, the machine-learned model is configured to further: identify properties of the oligonucleotide sequence or target genes that are susceptible to off-target gene knockdown above a threshold.

In some embodiments, if the dose-responsive gene feature is determined to be associated with the oligonucleotide sequence, the machine-learned model is configured to further: identify one or more target loci of target genes susceptible to on-target gene knockdown above the threshold.

In some embodiments, if the dose-responsive gene feature is determined to not be associated with the oligonucleotide sequence, the machine-learned model is configured to further: identify one or more target loci of target genes susceptible to on-target gene knockdown above the threshold.

In some embodiments, if the dose-responsive gene feature is determined to not be associated with the oligonucleotide sequence, the machine-learned model is configured to further: predict on-sequence features of target genes that lead the oligonucleotides to being susceptible to off-targets. In some embodiments, on-sequence features of target gene that lead to the oligonucleotide to off-targets can include, but are not limited to: single or double mismatch genes, and the like.

In some embodiments, the method further comprises validating a second set of oligonucleotides by synthesizing the second set of oligonucleotides based on the machine-learned model, administering the second set of oligonucleotides to a second set of cells at different dosages, performing the functional genomic analysis operation on the second set of cells to quantify gene expression, and measuring a difference between the quantified gene expression and a predicted gene expression produced by the machine-learned model.

In some embodiments, the second set of oligonucleotides comprises 50 or fewer oligonucleotides, between 50 and 100 oligonucleotides, between 100 and 150 oligonucleotides, between 150 and 200 oligonucleotides, between 200 and 300 oligonucleotides, between 300 and 400 oligonucleotides, between 400 and 500 oligonucleotides, between 500 and 750 oligonucleotides, between 750 and 1000 oligonucleotides, between 1000 and 1500 oligonucleotides, between 1500 and 2000 oligonucleotides, between 2000 and 2500 oligonucleotides, between 2500 to 5000 oligonucleotides, or between 5000 to 10000 oligonucleotides. It should be noted that additional sets of oligonucleotides can be generated over multiple iterations using increasingly complex mutations from the initial oligonucleotide of the set of oligonucleotides (or mutations of earlier sets of oligonucleotides) for use in updating/retraining the machine-learned model in order to improve the performance of the machine-learned model. In some embodiments, this process is iteratively performed until a stop condition is satisfied.

In some embodiments, the stop condition comprises a below-threshold increase in predictive performance of the machine-learned model after a refining iteration. In some embodiments, the number of refining iterations ranges from 1 to 3, 3 to 5, 5 to 10, 10 to 15, 15 to 20, 20 to 30, 30 to 40, 40 to 50, 50 to 60, 60 to 70, 70 to 80, 80 to 90, or 90 to 100. In some embodiments, the number of refining iterations is more than 100. In some embodiments, the number of iterations is 1 iteration, 2 iterations, 3 iterations, 4 iterations, 5 iterations, 6 iterations, 7 iterations, 8 iterations, 9 iterations, 10 iterations, 11 iterations, 12 iterations, 13 iterations, 14 iterations, or 15 iterations.

In some embodiments, generating a “refined” machine-learned model comprises one or more of: updating parameters of the existing machine-learned model or model architecture; updating drop parameters by sparsity, elastic net, dropoff or other model penalizations; and updating the machine-learned model architecture, including updating the feature space of the model entirely. In some embodiments, updating the feature space of the model entirely is performed by changing how variates are encoded in features or how connections between features are modeled.

In some embodiments, the machine-learned model comprises one of: an Ising model, a Potts model, a hidden Markov model, a continuous random field model, and a directed acyclic graphical model.

In some embodiments, the machine-learned model comprises one of: a random forest classifier, a logistic regression, a linear regression, a neural network, a sparsity-driven convex optimization fit, and a support vector machine.

In some embodiments, characteristics of the machine-learned model include constraints and factors. In some embodiments, the machine-learned model includes a set of coefficients representative of the factors. In some embodiments, the coefficients are updated during refinement of the machine-learned model (e.g., when the model is being refit/retrained) based on in vivo, in vitro, in silico, or in situ assays, or combinations thereof.

In some embodiments, the method further comprises modifying the machine-learned model based on the measured difference between the quantified gene expression and a predicted gene expression produced by the machine-learned model.

In some embodiments, the method further comprises validating a third set of oligonucleotides by synthesizing the third set of oligonucleotides based on the machine-learned model.

In some embodiments, the method further comprises: administering the third set of oligonucleotides to a subject at different dosages, performing the functional genomic analysis operation on DNA or RNA isolated from cells of the subject to quantify gene expression.

In some embodiments, the method further comprises measuring a difference between the quantified gene expression and a predicted gene expression produced by the machine-learned model.

In some embodiments, the method further comprises modifying the machine-learned model based on the measured difference between the quantified gene expression and a predicted gene expression produced by the machine-learned model.

In some embodiments, the method further comprises generating a final set of oligonucleotides using the trained machine-learned model.

In some embodiments, the final set of oligonucleotides has one or more of the identified set of characteristics that result in on-target gene knockdown. In some embodiments, the final set of oligonucleotides has one or more of the identified set of characteristics that result in higher on-target gene knockdown and reduced or no off-target gene knockdown compared to the original first set of oligonucleotides.

In some embodiments, the final set of oligonucleotides comprise an IC50 value ranging from 100 nM to 10 M. In some embodiments, the final set of oligonucleotides comprise an IC50 value ranging from 0.1 μM to 1 μM.

In some embodiments, the final set of oligonucleotides target an RNA that has an RNA half-life ranging from 1 minute to 72 hours.

### 5.7. Threshold

In some embodiments, the machine learned model is configured to: identify properties of the oligonucleotide sequence or target genes that result in on-target gene knockdown below a threshold.

In some embodiments, the machine learned model is configured to: identify properties of the oligonucleotide sequence or target genes that are susceptible to off-target gene knockdown above a threshold.

In some embodiments, the machine learned model is configured to identify one or more target loci of target genes susceptible to on-target gene knockdown above a threshold.

In some embodiments, the machine learned model is configured to identify one or more target loci of target genes susceptible to on-target gene knockdown above or below the threshold.

In some embodiments, the machine learned model is further configured to determine an association between toxicity and the gene expression feature.

The “threshold” as used herein in gene expression, is a numeric boundary (e.g., expression value, fold change, p-value, etc.) used to identify parameters or markers that meet the criteria above or below the numerical value. Whether the threshold is set as above or below, is dependent on upon the parameter or marker (or dose-responsive gene feature) being tested against.

For example, when knocking out a gene, the threshold can be used to determine whether the gene has been successfully silenced or disrupted to a biologically meaningful value. This can include functional or expression-based thresholds to determine effectiveness of the knockdown.

In some embodiments, the threshold is a gene expression reduction threshold. In some embodiments, the gene expression reduction threshold measures how much the gene's expression is reduced. In some embodiments, the threshold is a reduction of gene expression by 10% or more. In some embodiments, the threshold is a reduction of gene expression by 20% or more. In some embodiments, the threshold is a reduction of gene expression by 30% or more. In some embodiments, the threshold is a reduction of gene expression by 40% or more. In some embodiments, the threshold is a reduction of gene expression by 50% or more. In some embodiments, the threshold is a reduction of gene expression by 60% or more. In some embodiments, the threshold is a reduction of gene expression by 70% or more. In some embodiments, the threshold is a reduction of gene expression by 80% or more. In some embodiments, the threshold is a reduction of gene expression by 90% or more. In some embodiments, the threshold is a reduction of gene expression by 95% or more.

In some embodiments, the threshold is an increase in gene expression. In some embodiments, the gene expression threshold measures how much the gene's expression is increased. In some embodiments, the threshold is an increase of gene expression by 10% or more. In some embodiments, the threshold is an increase of gene expression by 20% or more. In some embodiments, the threshold is an increase of gene expression by 30% or more. In some embodiments, the threshold is an increase of gene expression by 40% or more. In some embodiments, the threshold is an increase of gene expression by 50% or more. In some embodiments, the threshold is an increase of gene expression by 60% or more. In some embodiments, the threshold is an increase of gene expression by 70% or more. In some embodiments, the threshold is an increase of gene expression by 80% or more. In some embodiments, the threshold is an increase of gene expression by 90% or more. In some embodiments, the threshold is an increase of gene expression by 95% or more.

In some embodiments, the threshold relates to fold change in gene expression.

In some embodiments, the threshold is absolute gene expression cutoff. In some embodiments, the TPM is <0.5. In some embodiments, the threshold is absolute gene expression cutoff. In some embodiments, the TPM is <0.4. In some embodiments, the threshold is absolute gene expression cutoff. In some embodiments, the TPM is <0.3. In some embodiments, the threshold is absolute gene expression cutoff. In some embodiments, the TPM is <0.2. In some embodiments, the threshold is absolute gene expression cutoff. In some embodiments, the TPM is <0.1.

In some embodiments, the threshold is a protein level threshold. In some embodiments, the protein level threshold can be measured using western blot, ELISA, flow cytometry, and the like. In some embodiments, the threshold is 10% or more reduction in protein signal. In some embodiments, the threshold is 20% or more reduction in protein signal. In some embodiments, the threshold is 30% or more reduction in protein signal. In some embodiments, the threshold is 40% or more reduction in protein signal. In some embodiments, the threshold is 50% or more reduction in protein signal. In some embodiments, the threshold is 60% or more reduction in protein signal. In some embodiments, the threshold is 70% or more reduction in protein signal. In some embodiments, the threshold is 80% or more reduction in protein signal. In some embodiments, the threshold is 90% or more reduction in protein signal.

In some embodiments, the threshold is 10% or more increase in protein signal. In some embodiments, the threshold is 20% or more increase in protein signal. In some embodiments, the threshold is 30% or more increase in protein signal. In some embodiments, the threshold is 40% or more increase in protein signal. In some embodiments, the threshold is 50% or more increase in protein signal. In some embodiments, the threshold is 60% or more increase in protein signal. In some embodiments, the threshold is 70% or more increase in protein signal. In some embodiments, the threshold is 80% or more increase in protein signal. In some embodiments, the threshold is 90% or more increase in protein signal.

### 6. EXAMPLES

### 6.1. Example 1: a Dose-Response Model for Accurate Detection and Quantification of Transcriptome-Wide Gene Knockdown for Oligonucleotide-Based Medicines

In this study, a dose-response model (used interchangeably herein as “DoReSeq”, which stands for Dose-Response Model using Sequencing) was developed and tested for characterizing the level of gene expression dependent upon the concentration of an ASO or an siRNA and as detected by NGS at every dose-point. This study aimed to both identify which genes are knocked down (whether on-target or off-target), as well as to quantify both the level of knockdown and the confidence in such knockdown calls.

DoReSeq is composed of three parts: a kinetic model of dose-response, a noise model that describes biological and technical noise in next generation sequencing assays, and a Bayesian inference fitter.

A kinetic, dose-responsive model of mRNA knockdown was first constructed by an ASO. This model describes how the competition between pre-mRNA maturation and the ASO guided cleavage of the pre-mRNA leads to a dose-dependent time-evolution of the concentration of mature mRNAs. In the long-time limit, this model predicts that the concentration of mRNAs as a function of the dose reaches a steady-state that coincides with the Monod equation (also known the empirical Hill equation with n=1). Time between dosing and readout is a confounding variable, that is often chosen to be different in different experimental setups. Therefore time was incorporated into the dose-response model, which allowed DoReSeq to be used to fit the RNA decay rate and thus compare results of experiments with different readout times.

Second, the kinetic model was supplemented with a noise model of gene expression as measured by NGS. The noise model of DoReSeq was applied to the realm of nonlinear models as opposed to generalized linear models. Specifically, the kinetic model was used to parameterize the mean response of a gene i as a function of dose A (and time t). Noise was modeled using the Negative Binomial distribution that has a gene specific dispersion parameter ϕi and a mean parameter, given by the kinetic model and scaled by a sample determined scaling factor sα (equal to the total number of non-duplicate reads for the sample, where a is the sample index).

Third, the full power of Bayesian inference was used to detect dose responsive genes and quantify their response from DGE datasets. Previous work on the use of Bayesian inference for analyzing gene expression focused on obtaining maximum a posteriori (MAP) estimates of model parameters. However, the recent advances in Markov Chain Monte Carlo (MCMC) sampling strategies, as well as increasing compute power, allow this model to efficiently and quickly sample probability distributions of the DoReSeq model parameters across thousands of genes. As a result, DoReSeq was able to obtain not just the MAP estimates, but also credibility regions and p-values for the model parameters and other key observables.

The sample-specific parameters were treated: the scaling factor s_{α}, the time t_{α}, and the dose A_{α} as confounding/independent variables and x_{a,i} the raw number of reads of gene i in sample α as the dependent variable. MCMC was used to sample the DoReSeq model parameters for each gene i: (1) TPMi, the number of Transcripts per Million (TPM) at zero dose; (2) ϕi, the negative binomial distribution dispersion parameter; (3) mKDi, the maximum knockdown at high dose; (4) rIC50,i, the relative rIC50, i.e. the dose needed to achieve a relative decrease in expression, as compared to mKDᵢ, of 50%; and (5) δᵢ, the mRNA decay rate.

Using synthetic data, it was shown that DoReSeq could be used to robustly identify dose-responsive genes and recover the model parameters used to synthesize the data. By analyzing synthetic data, an interesting caveat was discovered: for genes with weak dose-response, the mKDi and rIC50 space contains one stiff and one sloppy direction. The sloppy direction makes it hard to simultaneously fix the value of both m KDᵢ and r IC50. In Bayesian inference, this property results in credibility regions being inflated along the sloppy direction. Somewhat counterintuitively, it was found that the knockdown at a specific dose (within the range of doses probed in the experiment) is a stiff observable. Therefore, in this regime DoReSeq was still able to robustly identify genes with weak dose-response, even though characterizing their response in terms of mKDᵢ and rIC50 is poor without additional data to pin down the sloppy direction.

Finally, DoReSeq was applied to experimental DGE data. As this dataset was acquired at a single time point, we were not able to fit δi. Using D oReSeq, 203 dose-responsive genes were identified, containing one on-target response and 202 off-target responses (out of 8005 genes with good sequencing data) and obtained tight estimates of the model parameters for those genes.

By combining Negative Binomial distribution of gene expression noise with the dose-response model and Bayesian inference, the data fitting method accurately maps gene expression data to a family of most probable dose-response curves.

DoReSeq's strength lies in its capacity to integrate large collection of DGE datasets pertaining to dose response analysis, especially beneficial in scenarios where a substantial number of control samples are available. It significantly increasing the confidence in the design and discovery of precision OBMs by providing a statistically sound method of identifying off-target liability.

### 6.1.1. The DoReSeq Kinetic Model of Dose Response

This section describes construction of the DoReSeq model that describes the competition between pre-mRNA maturation and ASO guided cleavage of the pre-mRNA on recruiting RNaseH. A single gene was focused on and considered the population of three species as shown FIG. 1. The population of pre-mRNAs is denoted by T, the population of pre-mRNAs bound to an ASO by Tt, and the population of mature mRNAs by M. In this model, the ensemble of cells transcribes pre-mRNAs at a fixed, mean rate β. The pre-mRNAs can either mature, which occurs with rate T*, or become bound by an ASO, which occurs at the rate T αA where A is the ASO concentration and α is the rate constant. Once an ASO-pre-mRNA complex is formed it can either be cleaved, which occurs at rate T*κ, or become a mature mRNA which occurs at rate T*γ. Finally, the mature RNAs decay at rate Mδ. In constructing the kinetic model, a simplifying assumption was made that the ASO is regulating gene expression exclusively through RNaseH mediated knockdown and that the ASO has no effect on the transcription rate β, the maturation rate γ, nor the mRNA decay rate δ. As a consequence, the description of biological processes such as ASO induced gene up-regulation is beyond the scope of the kinetic model. The kinetic model has several key features.

One of the key results of the model is that knockdown can saturate to a finite, non-zero value, even at very high ASO concentrations. This feature emerges naturally because the model captures two critical biological mechanisms, in the tradition of simple systems-level models of biological processes. First, is a mechanism where the target—site may not be available for productive cleavage that leads to gene knockdown-because ASOs site, etc. and therefore, to ASO-guided cleavage. Second, a simplifying assumption was made that the maturation rate of state T (regular pre-mRNAs) and state T* (ASO-bound pre-mRNAs) is identical. This assumption is reasonable because the ASO: RNA bound state is a very local stretch (10-20 Bp long) of the pre-mRNA, and the fate of the pre-mRNA without RNaseH activity is typically determined at a significantly longer length scale. Consequently, even at high ASO concentrations, where almost all pre-mRNAs become bound by ASOs, there is still a competition between the maturation and RNaseH-mediated-cleavage of bound pre-mRNAs. The fraction of bound pre-mRNAs that can be cleaved before they become mature sets the limit on the maximum knockdown.

In constructing the model, the simplification was made that RNAs are transcribed at a steady rate. While this assumption is incorrect at a single-cell resolution for which transcription is known to occur in bursts, this a reasonable assumption for a large population of cells for which such transcriptional bursts average to an effective birth rate of pre-mRNA. The simplification was made that that the off-rate of ASO is negligible compared to all other rates, meaning, there is no T*→T transition in the model. This assumption is justified for bridged-nucleic acid ASOs for which the melting temperature (of 16-20 mers) is rather high (for standard gapmer designs of phosphorothioate using Locked Nucleic Acids (LNA)). It is easy to extend the model to include off-rate of ASO. In DGE experiments, timescale of readout is important such feedback typically happen at a longer timescale compared to enzymatic activity. The model includes kinetic effects to accommodate this biological challenge—the RNaseH-mechanism driven direct knockdown effects are easier to separate compared to indirect effects from ASO toxicity at earlier time-points (e.g., 5-6 hrs). However, at this time-point one may not reasonably expect for RNA knockdown effects to have reached steady state values because cellular uptake of ASOs and RNA half-life timescales are also in hours. The kinetic (time-dependent) dose-response model is seen as a major advantage of our work. To summarize, this model only includes first-order interactions for RNaseH-mediated knockdown.

The processes described above result in the kinetic equations described herein. The kinetic equations of the model and their properties are described below.

$$\frac{d}{dt} [ \begin{matrix}T \\ M \\ {T}^{*}\end{matrix} ] = [ \begin{matrix}- ( γ + α A ) & 0 & 0 \\ γ & - δ & γ \\ α A & 0 & - ( κ + γ )\end{matrix} ] [ \begin{matrix}T \\ M \\ {T}^{*}\end{matrix} ] + [ \begin{matrix}β \\ 0 \\ 0\end{matrix} ] \qquad ( Equation ( 1 ) )$$

To analyze the implications of these rate equations, the steady state populations were found in the absence of ASO treatment and then these steady-state populations were used as the initial condition for time evolution following ASO treatment.

To find the initial, steady-state populations T₀, M₀, and T₀*, the LHS was set of eq. (1) to zero and also set the ASO concentration A=0 to obtain:

$${T}_{0} = β / γ {M}_{0} = β / δ {T}_{0}^{*} = 0 , \qquad ( Equation ( 2 ) )$$

$$T ( t ) = \frac{β}{A α + γ} ( 1 + \frac{A α {e}^{- t ( A α + γ )}}{γ} ) , \qquad ( Equation ( 3 ) ) ,$$

$$M ( t ) = \frac{A αβκ {e}^{- t ( A α + γ )}}{( A α + γ ) ( A α + γ - δ ) ( A α - κ )} - \frac{A αβκ {e}^{- t ( γ + κ )}}{( A α - κ ) ( γ + κ ) ( γ - δ + κ )} + \frac{A αβκ {e}^{- t δ}}{δ ( A α + γ - δ ) ( γ - δ + κ )} + \frac{β γ ( A α + γ + κ )}{δ ( A α + γ ) ( γ + κ )} , \qquad ( Equation ( 4 ) ) ,$$

$${T}^{*} ( t ) = A α β ( \frac{κ {e}^{- t ( γ + κ )}}{γ ( A α - κ ) ( γ + κ )} - \frac{A α {e}^{- t ( A α + γ )}}{γ ( A α + γ ) ( A a - κ )} + \frac{1}{γ ( A α + γ ) ( γ + κ )} ) . \qquad ( Equation ( 5 ) )$$

Now, the focus included population of mature mRNAs, as that is the quantity that is being measured experimentally. Taking the long-time limit, the steady state population was found as shown below:

$$M ( t \to ∞ ) = \frac{β}{δ} ( 1 - \frac{κ}{γ + κ} \frac{A α}{A α + γ} ) . \qquad ( Equation ( 6 ) ) .$$

From this expression, the maximum knockdown (saturation effect) can be read off at high ASO concentration to be:

$$mKD = \frac{M ( t \to ∞ , A \to ∞ )}{{M}_{0}} = \frac{γ}{γ + κ} . \qquad ( Equation ( 7 ) )$$

This expression highlights that the maximum knockdown is controlled by the competition of mRNA maturation rate γ and the ASO guided cleavage rate κ. For fitting experimental observations, it was convenient to reparameterize the response using mKD and rIC50=γ/α:

$$R ( t \to ∞ , A ) = \frac{M ( t \to inf )}{{M}_{0}} = 1 - \frac{A ( 1 - mKD )}{A + {rIC}_{50}} \qquad ( Equation ( 8 ) )$$

Next, the present inventors explored how M(t) approaches the steady state. The expression for the time dependence of M(t) in equation Eq. (5) is rather complicated, involving four different terms, one static and three exponential decays controlled by three different rates: Aα+γ, γ+κ, and δ. To gain intuition, the constituent rates Aα, γ, κ, and δ were first looked at. In order for knockdown to occur κ, Aα≥γ, otherwise there will not be appreciable flow T→T*→cleavage. On the other hand, it was expected to be in the regime δ<<γ, which is usually appropriate for genes in eukaryotic cells in which mRNA maturation time is much shorter than mRNA half-life. Hence it was expected to be a short transient associated with the fast rates Aα+γ and γ+κ in which the populations T(t) and T*(t) reach their steady-state values, followed by a long decay associated with rate δ over which M(t) reaches its steady-state value. Which is indeed what was observed as shown in FIG. 2, as for the case δ=0.2γ.

Next the following simplifying assumption was made which was that δ<<γ, i.e. the initial transient was ignored in which the populations T(t) and T*(t) reach their steady-state values. Under this assumption, the two fast decaying terms were dropped and simplified the denominator of the remaining term of M(t) in Eq. (5) to obtain the response function:

$$R ( t , A ) = \frac{M ( t )}{{M}_{o}} \approx 1 - \frac{A ( 1 - mKD )}{A + {rIC}_{50}} ( 1 - {e}^{- t δ} ) , \qquad ( Equation ( 9 ) ) ,$$

where the same parametrization was used in terms of mKD and rIC50 as in Eq. (8). In FIG. 2 the response function obtained was compared from the exact time evolution Eq. (5) and from the approximate time evolution Eq. (9). It was observed that the two curves were almost identical, except for the small initial transient that can be seen in the zoomed view plotted in the inset.

In FIG. 3, plot response curves were plotted as a function of dose for various times using both the exact solution of Eq. (5) as well as the approximate solution Eq. (9). It was observed that if time was fixed, the response increases with dose saturating at high dose. At the same time, it was observed that as time increases so does the response until the steady state is reached at long times. Finally, it was observed that for δ≤0.2γ, exact and approximate solutions of the kinetic model are almost identical. Eq. (9) is a simple and convenient expression for parameterizing the response of gene expression to ASO treatment.

### 6.1.2. The DoReSeq Noise Model

In order to connect the dose response model to the noise in gene expression, the following expression was introduced for the expected mean number of counts of gene i in sample α:

$$μ = [ \frac{{s}_{α} {TPM}_{i}}{{10}^{6}} ] [ 1 - \frac{{A}_{α} ( 1 - {mKD}_{i} )}{{A}_{α} + {rIC}_{50 , i}} ( 1 - {e}^{- {t}_{α} {δ}_{i}} ) ] , \qquad ( Equation ( 10 ) ) ,$$

where sα is the scaling parameter for sample α; A_{α} is the ASO dose in sample α; tα is the time at which sample α was read out; TPMᵢ is the zero-dose transcripts per million value for gene i; mKDᵢ, rIC50,ᵢ, and δᵢ are the gene-specific DoReSeq parameters as described in the previous section. In this analysis s_{α}=N_{α} to be the total number of non-duplicate reads of all genes in sample α. The term in the first bracket on the RHS of Eq. (10) is the expected number of counts for gene i at zero dose, while the term in the second bracket is the fractional decrease in the counts induced by the treatment. In the following, sα, Aα, and tα will be treated a confounding/independent variables, and TPMi, mKDi, rIC50,ᵢ, and δᵢ as fit parameters.

The Negative Binomial distribution was used to described the noise in the NGS data, where the noise can be both biological or technical in nature. To simplify notation, Negative Binomial distribution PNB (x|μ, r) was parameterized using the mean, μ, and the dispersion parameter, ϕ. This parametrization is connected to the more standard parametrization in terms of p and r via

$$p = \frac{1}{1 + μ ϕ} r = \frac{1}{ϕ} . \qquad ( Equation ( 11 ) ) ,$$

where x_{α,i} is the number of reads of gene i in sample α and ϕi is the gene-specific dispersion parameter. In other words, Eq. (14) says that that the distribution of x_{α,i} is the negative binomial distribution with mean set by the expected number of reads from Eq. (10) and width set by the gene specific dispersion parameter ϕi. Further, in order to determine the expected number of reads, its necessary to have the values of the confounding/independent variables s_{α}, A_{α}, and t_{α} for sample α which are derived from the experimental data as well as the values of the fitting parameters TPMi, mKDi, rIC50,i, and δi. For the case where all measurements were carried out at the same time after treatment, the fitting parameter δi can be dropped and the expression (1−e^{-taδi}) set to unity.

### 6.1.3. DoReSeq Fitting Using Bayesian Inference

The scheme for fitting dose response using Bayesian inference is described herein.

The goal was given dose-dependent experimental data to find the probability distribution of the fit parameters [symbol] ={TPMᵢ, mKDᵢ, TIC_{50,i}, δ_{b} ϕᵢ}:

$$P ( {ℱ}_{i} \vert \{ {x}_{α , i} \} , \{ {𝒞}_{α} \} ) = \frac{P ( \{ {x}_{α , i} \} \vert {ℱ}_{i} , \{ {𝒞}_{α} \} ) P ( {ℱ}_{i} )}{\int d {ℱ}_{i} P ( \{ {x}_{α , i} \} \vert {ℱ}_{i} , \{ {𝒞}_{α} \} )} , \qquad ( Equation ( 15 ) )$$

where {Ca}={s_{α}, dose_{α}, t_{α}} is the set of confounding/independent variables. Eq. (14) was used to define the likelihood

$$P ( \{ {x}_{α , i} \} \vert {ℱ}_{i} , \{ {𝒞}_{α} \} ) = {\prod }_{i} {P}_{NB} ( {x}_{α , i} \vert {ℱ}_{i} , {𝒞}_{α} ) . \qquad ( Equation ( 16 ) )$$

With the parametrization, the mean and variance of the negative binomial distribution are:

$${\sum }_{x = 0}^{∞} {xP}_{NB} ( x \vert μ , r ) = μ , \qquad ( Equation ( 12 ) )$$

$${\sum }_{x = 0}^{∞} {( x - μ )}^{2} {P}_{NB} ( x \vert μ , r ) = μ + {μ}^{2} ϕ . \qquad ( Equation ( 13 ) )$$

Next, a model was introduced for noise in the number of reads. The following equation is provided below:

$$P ( {x}_{α , i} \vert {s}_{α} , {A}_{α} , {t}_{α} , {TPM}_{i} , {mKD}_{i} , {rIC}_{50 , i} , {δ}_{i} , {ϕ}_{i} ) = {P}_{NB} ( {x}_{α , i} \vert [ \frac{{s}_{α} {TPM}_{i}}{{10}^{6}} ] [ 1 - \frac{{A}_{α} ( 1 - {mKD}_{i} )}{{A}_{α} + {rIC}_{50 , i}} ( 1 - {e}^{- {t}_{α} {δ}_{i}} ) ] , {ϕ}_{i} ) , \qquad ( Equation ( 14 ) )$$

For the prior P( [symbol] ), box distributions were used with reasonable range for each of the fit parameters:

$$0.001 < {TPM}_{i} < 50000 , \qquad ( Equation ( 17 ) )$$

$$0.001 < {mKD}_{i} < 1 , \qquad ( Equation ( 18 ) )$$

$$0.001 < {rIC}_{50 , i} < 15 , \qquad ( Equation ( 19 ) ) ,$$

$$0.001 < {ϕ}_{i} < 10 , \qquad ( Equation ( 20 ) )$$

$$0.04 < {δ}_{i} < 1 [ 1 / hours ] , \qquad ( Equation ( 21 ) )$$

where δi is the only quantity with units and it was measured in inverse hours as indicated.

In order to efficiently explore the five dimensional space of fit parameters (four dimensional in case time-dependent data is not available) Markov chain Monte Carlo (MCMC) was used to sample the numerator of the RHS of Eq. (15). The output of MCMC is a list of samples: 5-dimensional (4-dimensional if there is no time-dependence) vectors that define a set of models. The vectors sample the probability density defined by Eq. (15)—there are more vectors clustered in the regions where P( [symbol] i|{xα,i}, {Cα}) is large and fewer in the regions where it is small. The sampling was performed using the emcee python package.

To turn the list of vectors sampled by MCMC into a probability density, there is a need to generate a normalized histogram. The focus was on constructing three types of histograms: (1) histograms of the parameters that specify the noise distribution FDS={TPMi, ϕi}; (2) histograms of the dose response parameters [symbol] _{DR}=data or [symbol] _{DR}={mKDi, rIC50,i} for the case there is only data at a single time-point; (3) histograms of the observable KD_{dose,time}, the knockdown at fixed dose and time. The histograms were used to obtain the maximum a posteriori (MAP) estimates of the fit parameters, credibility regions containing 50%, 95%, and 99% of the probability, and p-values for KD_{dose, time} to be above a thresh old. From the credibility contours, “error bars” were also obtained which correspond to the extent of the credibility contour in the cardinal directions. The three analysis that were performed are now described.

The noise distribution histograms provide us with an estimate of the fit parameters [symbol] DS={TPMi, ϕi}, which are crucial for identifying poorly-behaved genes, i.e. genes that have a low number of reads (e.g. TPMi<10) or large variability (e.g. ϕi>0.2). This is especially important with NGS data which includes thou-sands of genes (roughly 25,000 for the experimental DGE dataset) where it is infeasible to manually process the data gene-by-gene. Additionally, noise distribution histograms are helpful for interpreting dose-response analysis-they provides a basis for understanding why certain genes have larger credibility intervals than others when fitting dose response.

The dose-response histograms provide us with the data for quantifying the crucial property that we want to know: the dose-response parameters. Below, it is shown that the probability density function P(F) is not separable, and therefore it is important to analyze it as a joint probability distribution. Further, it was shown that P(F) contains a sloppy direction, which makes it hard to constrain the value of mKDᵢ and rIC_{50,i} simultaneously for weakly dose-responsive genes.

Finally, the fixed-dose, fixed-time response histograms combine the three dose-response fit parameters FDR={mKDi, rIC50,i, δi} into a single, biologically significant observable KDdose,time. KDdose, time is helpful for predicting the outcome of a particular treatment: e.g. how much knockdown can one expect at at a fixed treatment dose? Further, it was shown that KDdose,time is a stiff observable that makes it possible to identify strongly-, weakly-, and none-dose-responsive genes, e.g. using a p-value.

Then, the next step was to randomly sample the negative binomial distributions. In order to visualize the simulated data, the synthetic values of raw number of reads xαi was converted to synthetic TPM (transcripts per million) using:

$${TPM}_{α , i} = [ \frac{{s}_{α} {x}_{α , i}}{{10}^{6}} ] ,$$

and plotted the results in FIG. 1(a-c) using blue circles. The ground truth was also plotted.

### 6.1.4. Bayesian Inference on Synthetic DGE Data

In this section, synthetic DGE data was generated using the noise model of Eq. (14) and then used DoReSeq Bayesian inference to analyze it. The parameters that were used to generate synthetic data are typical of the fitted values for these parameters on experimental DGE data that are described in the next subsection. The present subsection is structured as follows. First, it was described how the Bayesian inference machinery works by analyzing a small synthetic dataset with 3 genes, 102 samples at various doses, and no time dependence. Next, 72 random replicates of the dataset were generated and investigate replicate-to-replicate fluctuations. Then, it was investigated how the sensitivity of our analysis depends on the number of samples, dispersion parameter, raw number of reads, and the total number of samples. This sub-section was concluded by analyzing a synthetic dataset with time dependence.

### 6.1.4.1 Setting Up Bayesian Inference Machinery to Fit Synthetic Data

First, a synthetic dataset that contains 102 samples and three genes were considered. The dose values A_{α} for the samples consisted of:

$$\begin{matrix}90 & zero - dose samples\end{matrix} \qquad Equation ( 22 ) :$$

$$2 \qquad samples at dose \{ 0.625 , 1.25 , 2.5 , 5. \}$$

$$4 \qquad samples at dose 10$$

The scale factors sα for the samples were chosen at random from a uniform distribution between 106 and 107. The three genes were chosen to contain a strongly dose-sensitive gene mKD1=0, a weakly dose-sensitive gene mKD2=0.5, and dose-insensitive gene mKD3=1.0. The ground-truth values for the other model parameters of the three genes were chosen to be identical: TPMi=50, ϕi=0.05, and rIC50,i=2. To synthesize the number of raw reads xα,i the time dependence was dropped from Eq. 14 to obtain:

$$P ( {x}_{α , i} \vert {s}_{α} , {A}_{α} , {TPM}_{i} , {mKD}_{i} , {rIC}_{50 , i} , {ϕ}_{i} ) = {P}_{NB} ( {x}_{α , i} \vert [ \frac{{s}_{α} {TPM}_{i}}{{10}^{6}} ] [ 1 - \frac{{A}_{α} ( 1 - {mKD}_{i} )}{{A}_{α} + {rIC}_{50 , i}} ] , {ϕ}_{i} ) , \qquad ( Equation ( 23 ) ) ,$$

TPM as a function of dose curves:

$${TPM}_{i} ( {A}_{α} ) = [ \frac{{s}_{α} {TPM}_{i}}{{10}^{6}} ] [ 1 - \frac{{A}_{α} ( 1 - {mKD}_{i} )}{{A}_{α} + {rIC}_{50 , i}} ] , \qquad ( Equation ( 24 )$$

which appear in Eq. (23), for the three genes using cyan lines.

Next, for each gene i the distribution of the model fit parameters were sampled {TPMi, ϕi, mKDi, rIC50,i}, given by Eq. (15), conditioned on the synthetic raw number of reads xα,i. Specifically, sα, Aα, and xα,i, was fed along with the (un-normalized) probability defined by the numerator of Eq. (15), to the emcee Markov Chain Monte Carlo (MCMC) code. The code was setup to use 40 walkers. For each walker the first 1000 points were burned and the next 4000 points were recorded in order to obtain a total of 160,000 fit parameter samples for each gene. Here, each sample is a 4-vector composed of {TPMi,μ, ϕi,μ, mKDi,μ, rIC50,i,μ}, where μ=1, 2 . . . 160, 000 is the MCMC sample index.

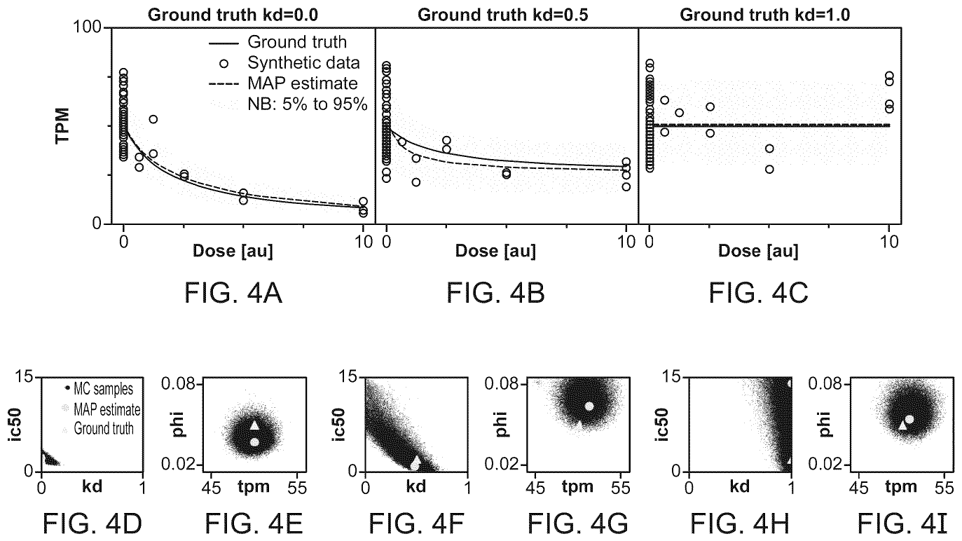

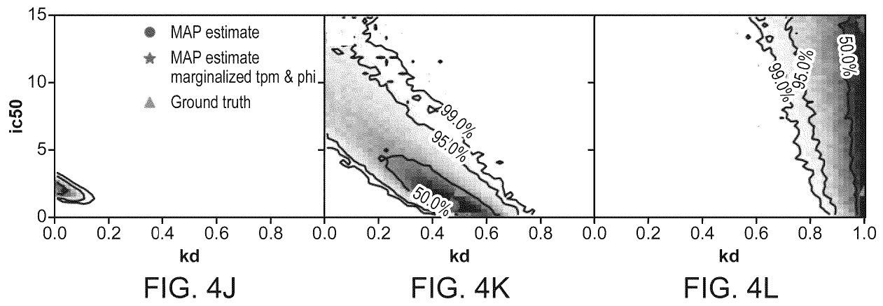

The MCMC samples were visualized in FIG. 4(d)-(i). In FIGS. 4(d) and (e), the MCMC sample was plotted for the gene with the strongest dose response. The ground truth value (i.e. the value of parameters we used to synthesize the data) was also plotted as well as the global MAP estimate (i.e. the MCMC sample with the highest probability, see Eq. (15)). FIGS. 4(f) and (g) pertain to the weakly dose-responsive gene while FIGS. 4(h) and (i) pertain to the dose non-responsive gene. Now several observations about the MCMC sampled distributions of fit parameters are made.

First, it was observed that for all three genes TPMi and ϕi are essentially independent of each other (see FIG. 4(e,g,i)), while mKDi and rIC50,i are strongly anti-correlated for the two dose responsive genes (see FIG. 4(d,f)). For the dose non-responsive gene, because mKDi˜1, the value of rIC50,i does not strongly affect the shape of the TPM vs dose curve and therefore observed that the distribution of rIC50,i almost matches the prior, being uniformly distributed between 0.001 and 15 (see FIG. 4(h). The anti-correlation between mKDi and rIC50,i implies the existence of a sloppy direction. This sloppy direction was identified as a simultaneous stretching out and lowering of the TPM vs dose curve by simultaneously increasing rIC50,i and decreasing mKDi. Under this transformation the quality of the fit is largely unaffected because the TPM vs dose curve does not substantially change for small and intermediate doses. Comparing FIGS. 4(d) and (f) it was observed that the sloppy direction is significantly bigger problem for weakly dose-responsive genes.

Second, it was observed that the fractional variance of MCMC samples of TPMi's is much smaller than that of ϕi's (see FIG. 4(e,g,i)). The underlying reason for this discrepancy is that it is much easier to estimate the mean of the negative binomial distribution as compared to its dispersion. Indeed, DESeq2 package recommends using a bootstrapping strategy to address this issue, in which the distribution of ϕi values from genes of simi-lar TPM's is used as a prior when fitting ϕi's. There is interest in estimating knockdown as opposed to putting a tight bound on the dispersion parameter ϕi and therefore we simply fit ϕi on a gene-by-gene basis.

However, in this case this fitting is strongly enhanced by having a large number (110) of zero-dose samples in our DGE datasets. Gene knockdown parameters mKDi and rIC50,i are focused on next. These are the key parameters of inter-Let us focus on the gene knockdown parameters mKDi and rIC50,i next. These are the key parameters of inter-marginalize over them. In FIGS. 4(j-l), the joint probability distributions were plotted for mKDi and rIC50,i which were obtained by histogram of the MCMC data. From the joint probability distributions, the marginalized MAP estimates were read off for mKDi and rIC50,i (marked with a star in FIGS. 4(j-l)) as well as the credibility regions (the boundaries of the 50%, 95%, and 99% credibility regions are marked with black lines in FIGS. 4(j-l)).

### 6.1.4.2 Gene Knockdown at a Fixed Dose: Using Bayesian Machinery on an Observable

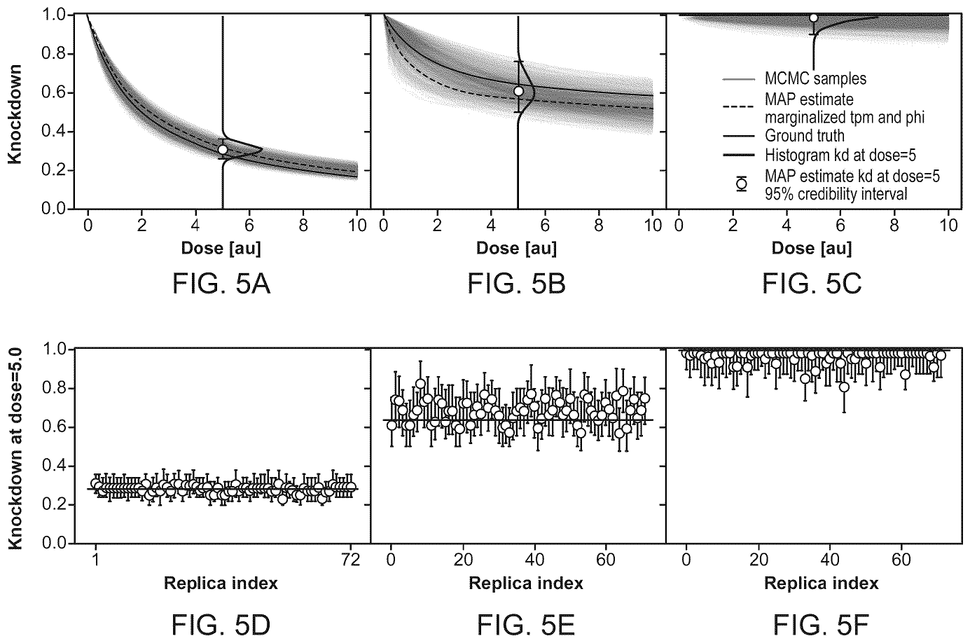

A useful alternative to estimating the fitting parameters is estimating the value of an observable. The observable that was focused on is the knockdown at a fixed dose. This observable was valuable for directly comparing the knockdown of different genes (e.g. to compare knockdown of off-target genes relative to the on-target genes). FIG. 5 shows how the model samples the probability distribution of knockdown at dose=5. The set of MCMC samples were taken of {mKDi, rIC50,i}μ (where μ is the MCMC sample index) and for each sample μ we compute the knockdown at dose=A=5:

$${KD}_{A , i , μ} = 1 - A \frac{1 - {mKD}_{i , μ}}{A + {rIC}_{50 , i , μ}} . \qquad ( Equation ( 25 ) )$$

Next, KDA,i,μ were histogramed to obtain PKD,A=5,i (KD5,i) the distribution of knockdown at dose=5. From this distribution the MAP estimate of KDA,i was found and the 95% credibility interval (see FIG. 5(a-c)). Finally the procedure was repeated on 72 replicas of the numerical experiment and plot the resulting MAP estimates of KD5,i and the 95% credibility intervals in FIG. 5(d-f). It was observed that that the credibility intervals are only a little wider for the weakly dose responsive gene (FIG. 5(e)) as compared to the strongly dose-responsive and the dose nonresponsive genes (FIG. 5 d) & (f)). At the same time, as expected, in almost all cases the 95% credibility intervals straddle the ground truth.

It was observed that the knockdown at dose=5 credibility intervals (FIG. 5(d-f)) were much narrower than the credibility regions of the maximum knockdown (FIG. 4(j-l)).

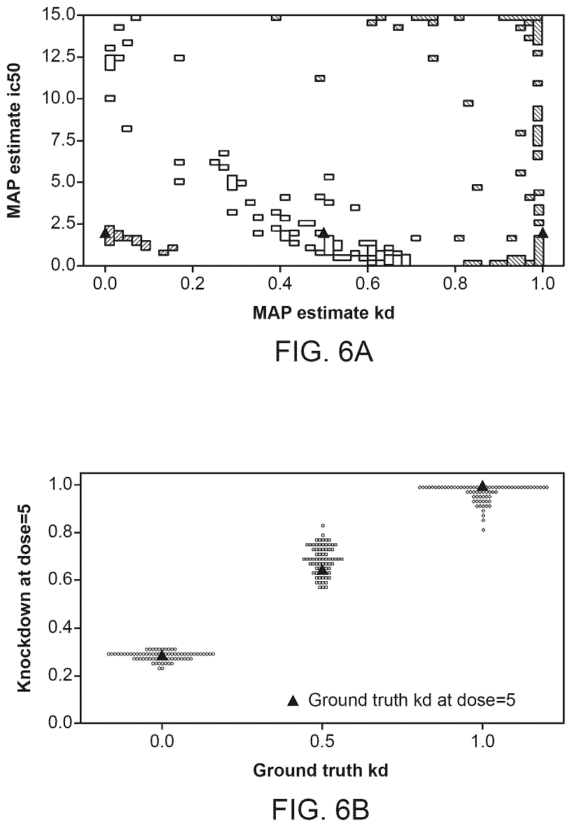

Similarly, the spread of MAP estimates of knockdown at dose=5 was much narrower than that of the maximum knockdown (see FIG. 6 and supplement for additional details).

This observation implies that KD5,i lies largely along the stiff direction of the {mKDi, rIC50,i} space and therefore we recommend using response at a fixed dose as a way to identify dose-responsive genes. To help identify dose-responsive genes, one further step was made: introducing a p-value for response of gene i at dose A. Specifically, the p-value measures the probability that KDA,I is above a threshold θ:

$${P}_{i} ( A ; θ ) = {\int }_{θ}^{1} {P}_{KD , A , i} ( x ) dx , \qquad Equation ( 26 ) ,$$

where a reasonable value for θ is 0.8. If the p-value for a certain gene is large, e.g. Pi (A=5; θ=0.8)>0.99, then it is very likely that the gene is not being knocked down. On the other hand, if the p-value is small, e.g. Pi (A=5; θ=0.8)<0.01, then it is very likely that the gene is being knocked down.

### 6.1.4.3 Dependence of Fit Quality on Parameters Used to Generate Synthetic Data

The fit quality depends on the parameters used to generate the synthetic data. In order to quantify the fit quality, 288 replicates were generated at each condition and compute PKD,A=5,i, the distribution of MAP estimates of knockdown at dose=5, as well as:

$$( ) ,$$

the median of the upper (lower) edge of the 95% credibility intervals. Four separate sweeps were performed in which we vary the ground truth value of one of the fit parameter at a time: mKDi, rIC50,i, ϕi and TPMi while the remaining parameters are held fixed, see FIG. 5(a-d). A sweep was also performed of the number of samples while holding the ground truth values of all the other DoReSeq parameters fixed, see FIG. 5(e). When sweeping the ground truth values of the fit parameters, each replicate consisting of 102 samples with doses prescribed by Eq. (22) and scale factors sa chosen from a uniform distribution between 106 and 107. On the other hand, when sweeping the number of samples, each replicate consists of an equal number of samples at each of the following doses: {0, 0.625, 1.25, 2.5, 5, 10} (e.g. when the number of samples is 12, there are 2 samples at each dose).

Several trends were observed. First, as the mean expected number of raw reads becomes small at strong knockdown, the variance in the number of raw reads shrinks as prescribed by Eq. (13) and therefore it was expected that the quality of fit will improve. The relationship between gene expression and the width of the negative binomial distribution in FIG. 4(a) was then observed, where the green shaded region denoting the width of the negative binomial distribution becomes narrower as gene expression is knocked down. The relationship between stronger knockdown and higher quality fitting can be seen in FIG. 5 a) at low ground truth values of mKDi as well as FIG. 5(b) at low ground truth values of rIC50,i.

Second, if there is very little knockdown, the quality of the fit will also improve because the prior encoded by Eq. (18) will squeeze the distribution of fitted knockdowns from above. This effect can is observed in FIG. 5(a) as the ground truth values of mKDi→1.

Third, for larger values of the ground truth dispersion parameter ϕi the synthetic data has larger variance and hence the quality of the fit degrades. This trend is observed in FIG. 5(c), where fitting becomes unreliable for ground truth ϕi≥0.256.

Fourth, DGE is expected to lose precision when the raw read counts become small. This loss of precision is a property of the Negative Binomial distribution, in which the ratio of the standard deviation to the mean:

$$\frac{σ}{μ} = \sqrt{ϕ + \frac{1}{μ}} , \qquad Equation ( 27 )$$

starts to grow rapidly for μ≤1/ϕ. In our case, this condition starts to set in for TPMi˜2−20 (where the range is set by the distribution of scale factors 106≤sα≤107). We indeed observe this trend in FIG. 5(d). A slow degradation was seen of the quality of fit as the ground truth value decreases to TPMi=16. At TPMi=8 the fit becomes significantly worse, and for TPMi≤4 the fit becomes unreliable as the median value of the upper edge of the credibility intervals sharply jumps up and the distribution of MAP estimates acquires weight at KD5,i=1.

Fifth, it was expected that the fit quality degrades as the number of samples decreases. This trend was observed in FIG. 5(e).

### 6.1.4.4 Time Dependence

In this subsection, the time dimension were added to synthetic data. Specifically, a dataset was synthesized that contains samples measured at 2, 4, 8, and 16-hour marks. At the 2, 4, 8, and 16 hour marks the dataset contains two samples at dose {0.625, 1.25, 2.5, 5} and four samples at dose 10. In addition, the dataset contains 90 zero-dose samples. As before, the scale factors were chosen for each sample at random from a uniform distribution between 106 and 107. Synthetic data was generated for four genes with δi={0.5, 0.25, 0.12, 0.6} [1/hours] and the ground truth values for the remaining fit parameters set to TPMi=50, ϕi=0.05, mKDi=0, and rIC50,i=2.

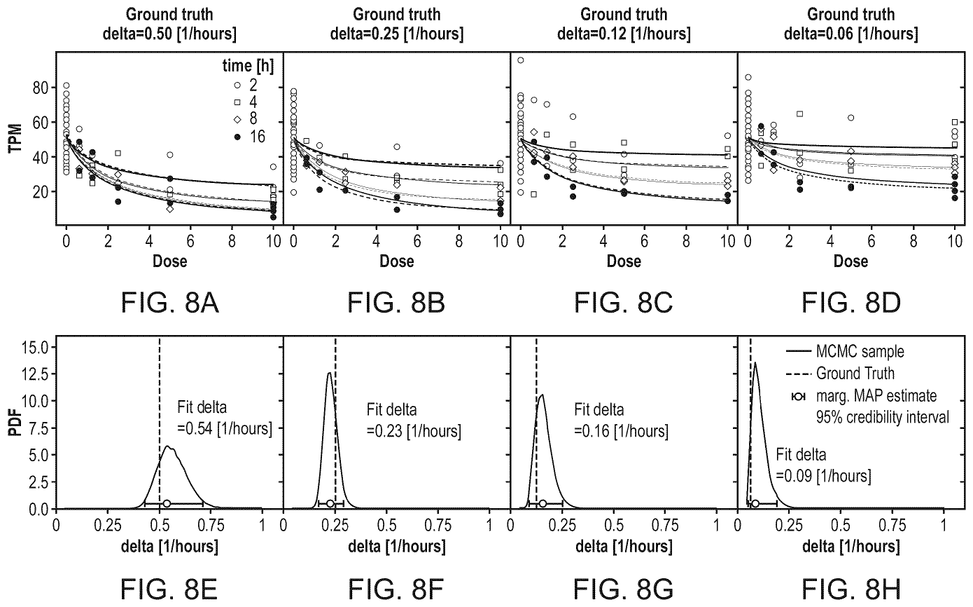

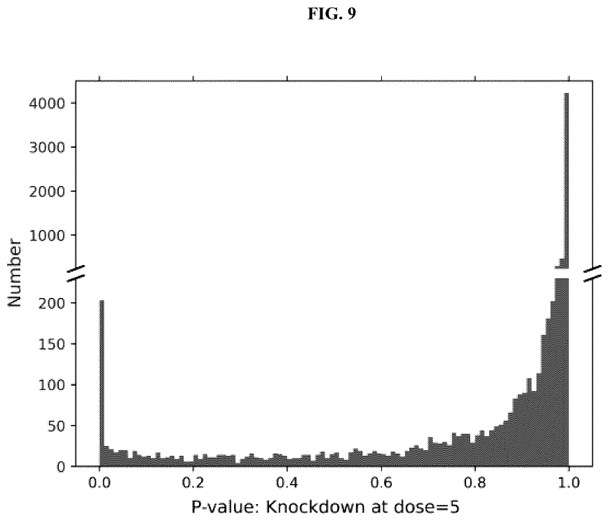

In FIG. 8(a-d) the time-dependent synthetic data was plotted that were going to fit with points and the corresponding ground-truth TPM vs. dose curves with solid lines.

Next, the Bayesian inference machinery was applied to fit the time dependent synthetic data. To assess the quality of the fit, we plot the TPM vs. Dose curves obtained from the global MAP estimate of the fit parameters in FIG. 8(a-d) with dashed lines. It was observed that in all cases the fits are quite close to the ground truth curves (solid lines). Further, in FIG. 8(e-h), the MCMC generated distributions were plotted of the fit parameter δ (i.e. the RNA decay rate) for the four different ground truth values of δ. From the MCMC distributions we also obtain the marginalized MAP estimates for δ as well as the 95% credibility intervals. It was observed that the MAP estimates (listed in the figure) are close to the ground truth values of δ and the credibility intervals straddle the ground truth.

In summary, the evidence presented herein in this subsection indicates that it should be possible to extract the RNA decay rate from experimental data. It is important to have sufficient quantity and quality of DGE data and to choose the measurement times so that there is considerable variation in the expression of the target gene (that is the measurement times are dictated by ˜1/δ).

### 6.1.4.5 Bayesian Inference on Experimental DGE Data

The goal of this subsection was to apply the machinery that is developed in the present disclosure to analyze an experimental DGE dataset. The aim was to both identify dose-responsive genes and quantify their response.

The dataset that was analyzed included of 121 samples, with 110 zero-dose samples, 2 samples at dose {0.625, 1.25, 5}, 1 sample at dose {2.5}, and 4 samples at dose {10}—all ASO doses are in micro-molar (μM) units. The total number of non-duplicate counts ranged from 0.49 to 8.93 million with an average of 3.22 million per sample.

8005 genes were selected, with zero-dose TPMi≥10, for analysis. The table of raw counts were included and the table of metadata.

In order to remove unwanted variance from plate to plate variations, it was noted that when applying the Bayesian machinery to experimental data we used a separate and independent TPM parameter for each plate.

Specifically, we replaced Eq. (14) with

$$P ( {x}_{α , i} \vert {s}_{α} , {A}_{α} , {t}_{α} , \{ {TPM}_{i , p ( α )} \} , {mKD}_{i} , {rIC}_{50 , i} , {δ}_{i} , {ϕ}_{i} ) = {P}_{NB} ( {x}_{α , i} \vert μ , {ϕ}_{i} ) , \qquad Equation ( 28 ) ,$$

$$μ = [ \frac{{s}_{α} {TPM}_{i , p ( α )}}{{10}^{6}} ] [ 1 - \frac{{A}_{α} ( 1 - {mKD}_{i} )}{{A}_{α} + {rIC}_{50 , i}} ( 1 - {e}^{- {t}_{α} {δ}_{i}} ) ] ,$$

where {TPMi,p(α)} is a collection of TPM values associated with gene i, with one value assigned to each plate p(α). In this case there were 8 plates and hence 8 values of {TPMi,p(α)} were fitted for each gene. In order to compare experimentally obtained TPM values across different plates, a normalization scheme was introduced where we normalize the experimentally observed TPM value, on a plate-by-plate basis, to the mean of the MAP estimates of TPM over the plates:

$${TPM}_{norm , i , p} = {TPM}_{obs , i , p} \frac{{〈 {TPM}_{MAP , i , p} 〉}_{p}}{{TPM}_{MAP , i , p}} . \qquad Equation ( 29 ) .$$

The mixed probability distribution method was implemented for attenuating outliers. In this method, the Negative Binomial distribution was implemented for inliers with a much wider Negative Binomial distribution for outliers. For the experimental dataset, it was found that attenuating the outliers was not particularly important and hence the data analysis that was reported here has this feature turned off.

In order to identify dose-responsive genes, the p-value of the knockdown was computed at dose=5 for each of the 8005 genes. The p-value was evaluated using Eq. (26) with threshold set to θ=0.8. The histogram of p-values, see FIG. 9, appears to be bimodal with a peak at small pvalues that corresponds to strongly dose-responsive genes and another peak at large p-values that corresponds to the dose-non-responsive genes.

Next, in order to better understand the characteristics of the genes in the two peaks of the p-value distribution, as well as genes in between, three representative genes were chosen with TPM˜50, one strongly dose responsive gene (p-value=0), one weakly responsive gene (p-value=0.123) and one dose non-responsive gene (pvalue=1). The results were plotted for all 203 strongly dose responsive genes and a samples of 100 weakly dose responsive genes and 100 dose non-responsive genes in the supplement.

To visualize the dose response, the normalized experimental TPM values were plotted (see Eq. 24) as a function of dose in FIG. 10(a-c). Superimposed on the experimental points are MAP estimates of TPM as a function of dose (Eq. 24 obtained using the plate-averaged value of TPM: TPMi→ [symbol] TPMMAP,i,p [symbol] p) as well as the MAP estimate of the dispersion encoded as the 5% and 95% quantiles of the negative binomial distributions. It was observed that for the strongly dose-dependent gene, both the experimental data and the MAP estimate decrease substantially with dose (see FIG. 10(a)). On the other hand for the weakly dose-dependent gene, the decrease with dose is much less pronounced FIG. 10(b)) and for the dose-independent gene no decrease in gene expression with dose is observed FIG. 10(c)).

To understand the quality of the fit, the probability distributions were looked at for the knockdown parameters (see FIG. 10(d-i)). It was observed that for the strongly dose-responsive gene the MAP estimates of mKD and rIC50 come with reasonably tight credibility regions (see FIG. 10(d)). For the weakly dose-responsive gene the credibility regions are much less tight (see FIG. 10(f)).

Finally for the dose-non-responsive gene the credibility region is tight on mKD but loose on rIC50 (which is expected when there is no knockdown, see FIG. 10(i)).

These observations are consistent with our analysis of synthetic data in subsection IVA1. We observe tight credibility intervals for knockdown at dose=5 for all three genes, see FIG. 10(e,g,i). This second observation is again consistent with our analysis of synthetic data, see discussion in Sec. IVA2).

The observations on both synthetic and experimental data indicated that response at fixed dose is a reasonable way to identify dose-responsive genes (given good experimental design: appropriately chosen dosing, readout time, and number of samples). Genes that are indeed dose responsive can be further analyzed by breaking down the response into parameters mKD and rIC50.

### 6.1.5. Results

Described herein includes a connection between DoReSeq kinetics and the Monod or the Hill equations. The Hill equation describes the equilibrium of a reversible chemical reaction that is completely unrelated to the kinetics that we describe in DoReSeq. In contrast, the Monod equation describes the rate of bacterial growth in the presence of a substrate limiting growth, which is much closer in spirit to the kinetics that we describe in DoReSeq. The fact that in the long-time, steady-state limit DoReSeq dose-response curve matches the Hill equation is purely a coincidence. However, both DoReSeq and the Monod equation described kinetics and therefore the similarity between the two is not a coincidence, although the details of the kinetics and mechanisms are completely different, e.g. in case of DoReSeq the rate of RNA production is maximal in the absence of ASO, while the rate of bacteria growth is zero in the absence of the rate limiting substrate in the Monod equation. These distinctions were pointed out to clarify the point of deviation in the work herein from the phenomenological methods of steady-state dose response fitting commonly used in ASO and siRNA fields.

One of the key features of DoReSeq kinetic model is capturing the “window-of-opportunity” phenomena—the maximum knockdown of gene expression that an ASO can achieve is not controlled by the concentration of the ASO alone, but rather by the competition between the maturation of the pre-mRNA and the rate at which the ASO-bound pre-mRNA is cleaved by RNase H1. Further, from the kinetic model it was found that the concentration of the mature mRNA exponentially decays in time to its long-time value, with the rate constant set by the mRNA decay rate.

Another advantage of the work described herein is that, unlike DE-Seq/DESeq2 methods, the full power of Bayesian inference was used. Instead of just identifying MLE/MAP points, we compute the whole distribution. This allows to construct p-values and credibility intervals that directly quantify how constrained our fit is by the available data. The additional computational cost for our method is quite reasonable, e.g., about 2 hours to process 8000 genes on a 72-core machine.

Being able to compute the credibility intervals provides drug developers in oligonucleotide space with improved quantification for evidence-based decision making on what makes a specific ASO or siRNA—for example, a time-course in DGE analysis across multiple doses can be used to measure on-target response and identify off-targets in parallel. For off-targets, the time course in-forms us on primary hybridization-mediated off-targets and cytotoxicity-mediated off-target (pathway modulation). Time is an important confounding variable. DoReSeq includes a model of kinetics and thus allows us to fundamentally analyze the time dependence of gene expression in response to dosing. Experimentally, capturing time dependence purely from DGE dose-time course is somewhat challenging because we need sizeable dose-time samples to constrain the RNA decay rate parameter δ. However, decay rates can be learned transcriptome wide in other assays. Using synthetic data, we performed a numerical experiment to show that DoReSeq is indeed able to capture time dependence for genes that show strong dose-response. The close agreement of DoReSeq analysis of synthetic and experimental single-timepoint data gives us confidence in the methods described herein.

In this study, gene knockdown was the primary focus. However, the Bayesian statistical approach is general and can be used with different, perhaps phenomenological, dose-response models. For example, we can empirically extend the dose-response model to gene up-regulation by allowing for mKD>1. Moreover, though we motivated the dose-response model with RnaseH-engaging ASO-mediated knockdown of RNA, with a focus on intronic-region targeting ASOs, it is far more general. Such generality is not uncommon in system-level modeling. Fundamentally, the model captures a “window of opportunity”—the target loci in the RNA is available for enzymatic activity for knockdown effects only within a limited timescale, and kinetic race between the events of competing regulatory processing of RNA and enzymatic activity of knockdown mechanism-of-action are important. For ASOs, we have argued that co-transcriptional events like exon-definition and splicing, RNA-binding protein (RBP) occupancy, RNA-secondary etc. are reasonable mechanisms by which the target loci is rendered less “target-able”. For siRNAs, events after nuclear to cytoplasmic transport—for example, RBP and translational machinery engagement, regulatory-RNA interactions etc. can create a similar window-of-opportunity. The model does not need to commit to any one mechanism in particular, but if the timescales of any such events compete with the rate at which RNAi-machinery can knockdown mature RNAs, empirically, targetability constraints will be observed for siRNAs too.

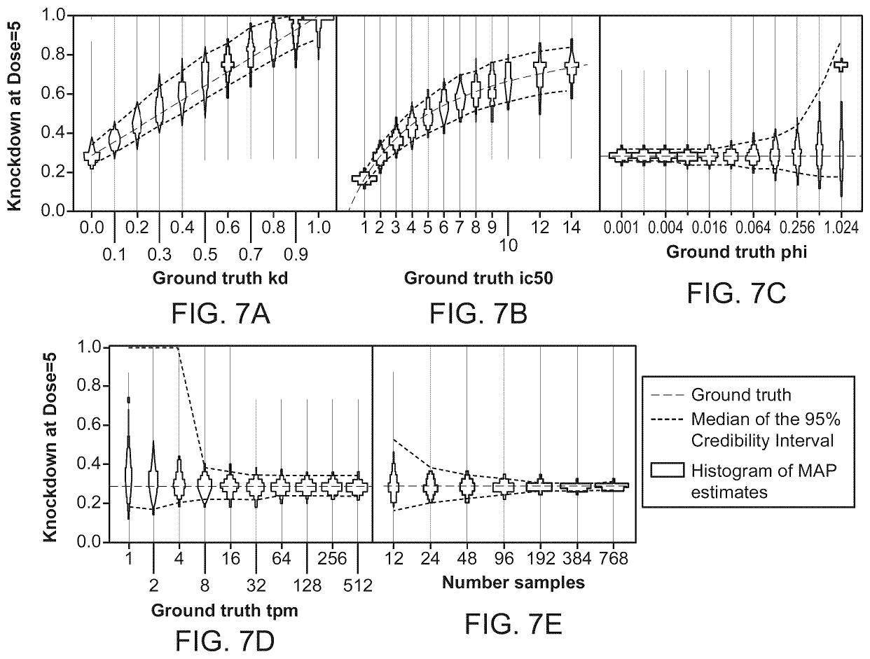

Finally, DoReSeq can be used to study experimental design. For example by fitting synthetic datasets with different number of samples, e.g. see FIG. 7E, one can figure out the statistical power of the experimental design (dosing regimen, number of replicates, depth of sequencing etc.) to detect and quantify dose-response reliably.

### 6.1.6. Conclusion

In this study, a method for fitting dose- and time-dependence of gene expression was described as measured by DGE, or other similar Next Generation Sequencing (NGS) methods.

A kinetic model was presented for ASO and RNaseH mediated knockdown of gene expression. Using our kinetic model, a time- and dose-dependent parametrization was derived for the response of gene expression to an ASO, Eq. 9. Next, a noise model for gene expression was constructed as measured by DGE (or other next generation sequencing methods). In the noise model, the number of reads of a particular gene is drawn from a Negative Binomial distribution with a time- and dose-dependent mean and a gene-dependent dispersion parameter. Specifically, the mean is constructed by multiplying our time- and dose-dependent parametrization for RNA concentration by a scale factor (i.e. the total number of reads for all genes). In order to fit gene expression data, the machinery of Bayesian inference and Markov Chain Monte Carlo was used to construct the probability distribution of fit parameters conditioned by the experimental data.

The Bayesian inference machinery was tested both on synthetic data as well as on experimental DGE data.

In both cases it was found that we can distinguish dose-responsive genes from dose-non-responsive genes using the P-value for response at fixed dose, given by Eq. (26). Genes were identified that have low p-values as dose-responsive. For the dose-responsive genes, the dose response can be further characterized by finding the Maximum A Pasteriori (MAP) estimates of three parameters: the maximum knockdown mKD, the relative IC50 rIC50 and, if sufficient time-dependent data is available, the RNA decay rate δ. mKD and rIC50 are the crucial two parameters, beyond the certainty of dose-responsiveness, as they enable us to determine if the knockdown is clinically relevant at the dose that was provided to set the on target effect. A long with MAP estimates we also compute credibility regions for these parameters, precisely quantifying how well the experimental data measures the values of these parameters. It was found that the credibility regions for these parameters are much broader for weakly dose-responsive genes, and much sharper for strongly dose-responsive genes, owing to the presence of a sloppy direction in the {mKD, rIC50} space.

A response at fixed dose can be used in order to identify strongly-, weakly-, and dose-non-responsive genes (using p-values) because this observable is more constrained—it is essentially orthogonal to the sloppy direction associated with increasing rIC50 (the relative IC50) and decreasing mKD (the saturating knockdown value).

The methods described herein allow for better quantification of on- and off-target of OBMs. Thus, the present methods will result in better engineered and safer OBMs, and avoids potentially dangerous off-target gene knockdown.

### 6.2. Example 2: Digital Gene Expression Profiling of Oligonucleotides Across Different Dosages

This study was performed to understand the correlation between dose-response of oligonucleotides at different dosages and their level of toxicity. A set of antisense oligonucleotides (ASOs) were categorized based on their cellular and liver toxicities, then profiled their gene expression with Digital Gene Expression (DGE).

Cellular toxicity was determined by ASO dose response in optical plates and measurement using the Promega RealTime-Glo™ MT Cell Viability Assay and RealTime-Glo™ Annexin V Apoptosis Assay kits to measure cell health and membrane disruption (as a proxy for apoptosis), respectively, through luminescence. Scoring of toxic or safe were based on comparisons between known toxic compounds and safe controls.

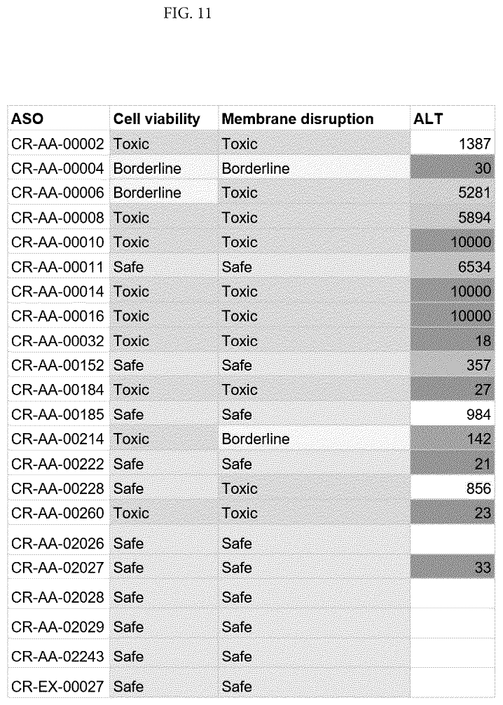

As shown in FIG. 11, liver toxicity was measured following dosing in rodents in triplicate and monitoring the blood for ALT levels, using standard thresholds for calling toxicity. The ASOs at different dosages were categorized in terms of cell viability and membrane disruption as “toxic”, “borderline”, or “safe”.

### 6.3. Example 3: Screening the RNA Expression Levels Across a Dose Response of

This study screened RNA expression levels after administering oligonucleotides at different dosages across a dose-response to generate a most likely curve of gene behavior.

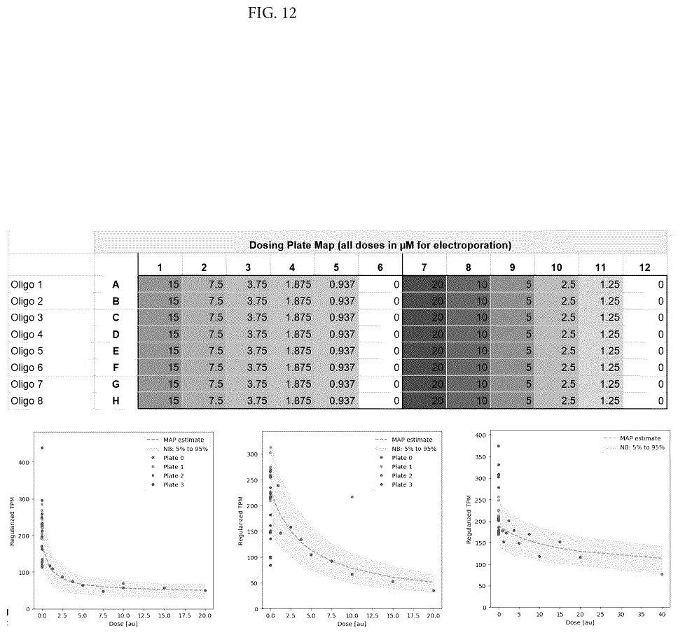

Cells were dosed with ASOs across a 10 point dose response (two offset 5-point dose responses). The RNA was later extracted and used to generate next generation sequencing libraries with the Lexogen QuantSeq-Pool Sample-Barcoded 3′ mRNA-Seq Library Prep Kit. The raw data was processed (deduplicated, deconvoluted, and mapped) through standard pipelines to generate TPM (transcripts per million) (shown for three genes across different ASO treatments). As shown in FIG. 12, this data was subsequently fed into the DoReSeq algorithm to generate a confidence interval for the most likely gene behavior (shading) for analysis.

### 6.4. Example 4: Determining which Genes are Enriched for Knock-Down by Toxic ASOs and Screening for Toxicity

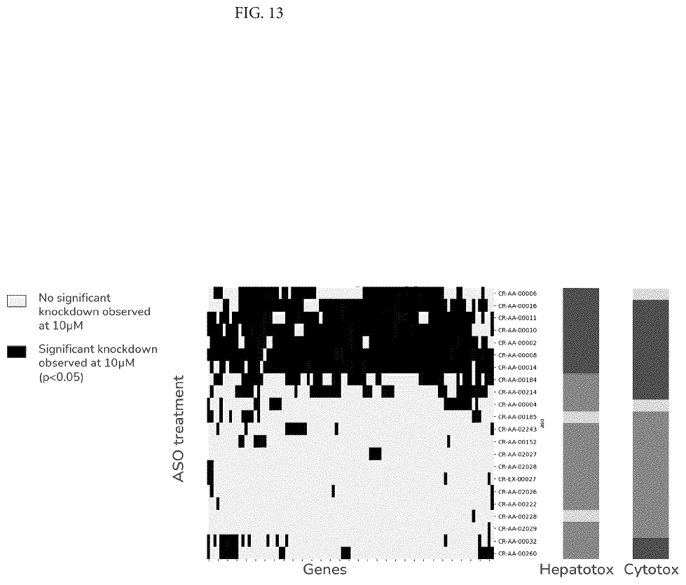

As shown in FIG. 13, gene response to both safe (green) and liver or cytotoxic (red) ASOs (shown on the right) was observed to determine which genes were more likely to be down-regulated by the toxic compounds. Each pixel in this image reflects whether DoReSeq found the gene to be significantly knocked down (black) or not (orange) by a given ASO.

### 7. EQUIVALENTS AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

1. A method for manufacturing oligonucleotide-based medicines, the method comprising:
a. administering a set of oligonucleotides to one or more cells, wherein each oligonucleotide within the set of oligonucleotides is administered at plurality of different dosages;
b. performing a functional genomic analysis operation on the one or more cells to quantify gene expression for each oligonucleotide dosage;
c. creating a training set by fitting a dose-responsive model to a dose-dependent response of each oligonucleotide on gene expression, wherein the dose-responsive model comprises
a kinetic model of dose-response,
a noise model of gene expression, and
a Bayesian inference model to detect and quantify dose-responsive gene features,
and wherein the dose-responsive model is configured to identify and characterize dose-responsive gene features dependent upon the dosage of each oligonucleotide of the set of oligonucleotides;
e. training a machine-learned model using the training set, the machine learned model configured to determine whether the dose-responsive gene feature is or is not associated with the oligonucleotide sequence,
wherein if the dose-responsive gene feature is determined to be associated with the oligonucleotide sequence, the machine-learned model is configured to further:
i. identify properties of the oligonucleotide sequence or target genes that result in on-target gene knockdown above a threshold;
ii. identify properties of the oligonucleotide sequence or target genes that are susceptible to off-target gene knockdown above a threshold; and/or
ii. identify one or more target loci of target genes susceptible to on-target gene knockdown above the threshold; and
wherein if the dose-responsive gene feature is determined to not be associated with the oligonucleotide sequence, the machine-learned model is configured to further:
identify one or more target loci of target genes susceptible to on-target gene knockdown or off-target gene knockdown above the threshold;
f. validating a second set of oligonucleotides by synthesizing the second set of oligonucleotides based on the machine-learned model, administering the second set of oligonucleotides to a second set of cells at different dosages, performing the functional genomic analysis operation on the second set of cells to quantify gene expression, and measuring a difference between the quantified gene expression and a predicted gene expression produced by the machine-learned model; and
g. modifying the machine-learned model based on the measured difference between the quantified gene expression and a predicted gene expression produced by the machine-learned model.

2. The method of claim 1, wherein the functional genomic analysis operation is a transcriptome analysis selected from digital gene expression (DGE), RNA sequencing (RNA-seq), tag-based RNA-seq (TAQ-seq), or a combination thereof.

3. (canceled)

4. The method of claim 1, wherein the dose-responsive gene features comprise on-target genes that are knocked down in response to each oligonucleotide and off-target genes that are knocked down in response to each oligonucleotide.

5. The method of claim 4, wherein the off-target genes comprise sentinel genes or one or more off-target loci within each off-target gene.

6. (canceled)

7. The method of claim 4, wherein the on-target genes comprise one or more on-target loci within each on-target gene.

8. (canceled)

9. The method of claim 1, wherein the dose-responsive gene feature is selected from: RNA-half life, polymerase occupancy, functional genomics features, RNA foundational model target gene loci, toxic off-target effects, and gene expression features comprising on-target genes, off-target genes, single-mismatch genes, or double mismatch genes.

10. (canceled)

11. The method of claim 1, wherein if the dose-responsive gene feature is or is not determined to be associated with the oligonucleotide sequence, the machine-learned model is configured to further: identify an association between the oligonucleotide sequence or target gene and one or more biomarkers measured from the one or more cells in response to each oligonucleotide administered at different dosages in the set of oligonucleotides.

12. The method of claim 11, wherein the one or more biomarkers is selected from: cytotoxicity, membrane toxicity, immunotoxicity, an effect that inhibits membrane fluidity, a membrane fusion and fission event, and an immune response.

13. The method of claim 1, wherein the method further comprises:
validating a third set of oligonucleotides by synthesizing the third set of oligonucleotides based on the machine-learned model,
administering the third set of oligonucleotides to a subject at different dosages, performing the functional genomic analysis operation on DNA or RNA isolated from cells of the subject to quantify gene expression, and
measuring a difference between the quantified gene expression and a predicted gene expression produced by the machine-learned model; and
modifying the machine-learned model based on the measured difference between the quantified gene expression and a predicted gene expression produced by the machine-learned model.

14. The method of claim 1, generating a final set of oligonucleotides. using the trained machine-learned model, wherein the final set of oligonucleotides has one or more of the identified set of characteristics that result in on-target gene knockdown.

15. (canceled)

16. The method of claim 14, wherein the final set of oligonucleotides comprise an IC50 value ranging from 0.1 to 1 μM or 100 nM to 10 μM or an RNA that has an RNA half-life ranging from 1 minute to 72 hours.

17. (canceled)

18. The method of claim 1, wherein the kinetic model of dose-response is configured to: analyze the time dependence of gene expression in response to the dose of each oligonucleotide of the set of oligonucleotides and parameterize the mean response of a gene as a function of dose (d) and time (t).

19. (canceled)

20. The method of claim 1, wherein the kinetic model comprises one or more assumptions, wherein the one or more assumptions is selected from:
the one or more cells transcribe pre-mRNA at a fixed mean rate β (transcription rate),
the pre-mRNA can mature or become bound by the oligonucleotide of the set of oligonucleotide;
when an oligonucleotide-pre-mRNA complex is formed, it can be cleaved or become a mature mRNA;
the mature RNA decay at a rate of Mδ;
the oligonucleotide of the set of oligonucleotides is regulating gene expression through RNAseH mediated knockdown;
the oligonucleotide has no effect on the transcription rate β, the mature rate γ, or the mRNA decay rate δ;
gene knockdown can saturate to a finite non-zero value; and
the maturation rate of state T (regular pre-mRNAs) and state T* (olignucleotide-bound pre-mRNAs) is identical.

21. The method of claim 1, wherein the method is configured to sample probability distributions of the dose-response kinetic model assumptions across at least thousands of genes.

22. The method of claim 1, wherein noise model is a negative binomial distribution comprising a gene-specific dispersion parameter ϕᵢ, and a mean parameter and is scaled by a sample-determined scaling factor sα comprising a total number of non-duplicate reads for a sample, wherein α comprises a sample index.

23. (canceled)

24. The method of claim 1, wherein the noise model is configured to identify biological and technical noise in the functional genomic analysis.

25. The method of claim 1, wherein the Bayesian inference model comprises a Bayesian inference fitter configured to detect dose-response genes and/or quantifying the dose response genes from the functional genomics analysis, and computes whole distribution to construct p-values and credibility intervals that directly quantify how constrained the fit is by the second training set.

26. The method of claim 1, wherein the dose-responsive model comprises fitting dose- and time-dependence of gene expression (e.g., time-dependence in response to dosing of the oligonucleotide); and
captures time dependence for genes that show positive dose-response to each oligonucleotide of the set of oligonucleotides, or
determines a maximum amount of knockdown of gene expression that the oligonucleotide achieves.

27. (canceled)

28. (canceled)

29. (canceled)

30. The method of claim 1, wherein the threshold is a reduction of on-target or off-target gene expression by at least 50%, at least 60%, at least 70%, at least 80%, or at least 90%.

31. (canceled)

32. (canceled)

33. (canceled)

34. (canceled)

35. The method of claim 1, wherein the set of oligonucleotides comprises one or more of: a set of aptamers, a set of oligonucleotide-aptamer conjugates, a set of antisense oligonucleotides (ASO), a set of anti-gene oligonucleotides, a set CpG oligonucleotides, a set single-guide RNAs, a set dual-guide RNAs, a set targeter RNAs, a set activator RNAs, a set of LNA oligonucleotides, a set of constrained ethyl (cEt) oligonucleotides, a set of adenosine deaminase acting on RNA (ADAR)-guiding RNA (AD-gRNAs), a set of steric-blocking oligonucleotides (SBOs), a set of antisense oligonucleotides that that recruit endogenously expressed ADARs, a set of antisense oligonucleotides that harness RNase H, a set of intron-targeted ASOs, and a set of exon-targeted ASOs.
